(19) United States
(12) Patent Application Publication
(10) Pub. No.: US 2024/0416144 A1
(43) Pub. Date: Dec. 19, 2024

(54) FLEXIBLE DELIVERY SYSTEMS AND METHODS FOR RADIOACTIVE SOURCES

(71) Applicant: HANGZHOU DASHTECH CO., LTD., Hangzhou (CN)

(72) Inventors: Xuetang WANG, Hangzhou (CN); Dingchen ZHU, Hangzhou (CN); Guangming FU, Hangzhou (CN); Xingxing LEI, Hangzhou (CN)

(73) Assignee: HANGZHOU DASHTECH CO., LTD., Hangzhou (CN)

(21) Appl. No.: 18/820,341

(22) Filed: Aug. 30, 2024

Related U.S. Application Data
(63) Continuation of application No. PCT/CN2023/079162, filed on Mar. 2, 2023.

(30) Foreign Application Priority Data
Mar. 3, 2022 (CN) 202210207077.0
Mar. 3, 2022 (CN) 202210208833.1
Aug. 26, 2022 (CN) 202211032981.9
Dec. 9, 2022 (CN) 202211586593.5
Jan. 5, 2023 (CN) 202310014707.7
Mar. 1, 2023 (CN) 202310182331.0

(51) Int. Cl. A61N 5/10

(52) U.S. Cl. CPC A61N 5/1007; A61N 2005/1009; A61N 2005/1012; A61N 2005/1024

(57) ABSTRACT

The present disclosure proposes a flexible delivery system and method for a radioactive source. The system includes a main body, a delivery channel, a flexible pushing wire, and a wire driving mechanism. The flexible pushing wire is disposed in the delivery channel, the delivery channel is configured to guide the flexible pushing wire to move forward and backward, the wire driving mechanism is capable of driving the flexible pushing wire to move reciprocally, and the flexible pushing wire is capable of driving seeds, or seed strain chains, or seed strand casings to a target position along the delivery channel, which can achieve high-precision positional control and implantation and solve the problem of drifting movement of the puncture needle and multi-channel implantation of radioactive sources.

### CROSS REFERENCE TO RELATED APPLICATIONS

This application is a continuation of International Patent Application No. PCT/CN2023/079162, filed on Mar. 2, 2023, which claims priority of Chinese Patent Application No. 202210207077.0, filed on Mar. 3, 2022, No. 202210208833.1, filed on Mar. 3, 2022, No. 202211032981.9, filed on Aug. 26, 2022, No. 202211586593.5, filed on Dec. 9, 2022, No. 202310014707.7, filed on Jan. 5, 2023, and No. 202310182331.0, filed on Mar. 1, 2023, the entire contents of each of which are hereby incorporated by reference.

### TECHNICAL FIELD

The present disclosure relates to the technical field of seed implantation, and in particular, to a flexible delivery system and method for a radioactive source.

### BACKGROUND

Radioactive seed implantation surgery involves a direct implantation of multiple radioactive seeds into a tumor through puncture for providing localized radiotherapy. This surgery is applicable to a wide range of cancers, including lung cancer, liver cancer, breast cancer, and prostate cancer. Radioactive seed implantation surgery has small incisions, minimal bleeding, and relatively few complications, yet effectively inhibits tumor growth.

The basic procedure of the radioactive seed implantation surgery involves taking a preoperative computed tomography (CT) scan and determining a puncture path and seed arrangement in a treatment planning system (TPS). According to a preoperative plan, multiple puncture needles are inserted into the tumor. This process can be aided by a puncture guidance template, ensuring that the spacing and orientation of the needles are consistent with the preoperative plan. After confirming via CT that all puncture needles have reached the target position, the surgeon then pushes multiple seeds through the channels created by the puncture needles into the tumor, completing the procedure.

However, the current procedure takes a long time, and doctors need to be in close contact with the seeds during implantation, exposing themselves to significant radiation. This greatly limits the application and promotion of this type of surgery. Consequently, seed implantation robotic systems have been developed. For example, the seed implantation surgical robot described in Chinese Patent Application No. CN 201910714054.7 features an automatic seed implantation device mounted at the end of the robot, enabling highly precise puncture and seed implantation. However, the seed implantation device is rigidly connected to the puncture needle throughout the procedure, meaning that seed implantation occurs immediately after the puncture. This alters the traditional manual surgery workflow, necessitating CT verification immediately after each puncture, which significantly increases the number of CT scans required for the patient and thereby their exposure to radiation. Additionally, because the puncture needle is rigidly connected to the seed implantation device and cannot be quickly detached, there is a higher risk of scratching the patient.

Existing seed implantation devices typically operate under CT or other imaging guidance. First, a puncture needle is inserted into the tumor to reach the target position. According to the preoperative TPS plan, a seed in the magazine is then pushed to an appropriate position using a push rod through the seed applicator. The puncture needle is then withdrawn to next position, the push rod retracted to the back side of the magazine, and next seed is automatically ejected. Then the next seed is pushed to the next position using the push rod. This process is repeated until the predetermined count of seeds is implanted into the tumor. However, because gravity, compression, blood flow, etc., may cause multiple seeds to be discretely distributed, it is easy to cause displacement of the seeds, which may lead to insufficient irradiation of the tumor by the seeds. Seeds may even move into normal tissues, causing embolism and severe surgical complications. With technological advancements, a plurality of seeds can now be spaced apart and connected using seed strand casing made of absorbable material, forming a seed strand. The seed strand can be arranged according to the preoperative TPS plan and implanted into the body in one step. However, conventional seed implantation devices cannot implant the entire seed strand automatically.

Chinese patent Application No. CN201810650275.8 proposes a device for implanting a radioactive seed strand. The technical solution is as follows. The needle assembly is connected via a detachable connection to the outer trocar of the needle. The radioactive seed strand is pre-placed inside, fixing the relative positions of the needle outer trocar and the push rod. By adjusting the positions of the rotating assembly, bracket, and cross-frame assembly, the needle outer trocar is aligned with the tumor and moved downward so that the piercing end of the needle outer trocar enters the specified position in the tumor. The positioning bolt is then loosened, the drive motor is activated, and the gears drive the rack vertically upward, thereby moving the needle outer trocar upward relative to the push rod, and implanting the radioactive seed strand into the tumor in one motion. While this method can achieve the implantation of the seed strand, the Chinese patent Application still has the following deficiencies: 1) the process of placing the seed strand still requires manual operation, posing a radiation risk and complicating the procedure; 2) the type of the seed strand and the spacing between adjacent seeds cannot be adjusted based on the characteristics of the tumor and the needs of the surgery; and 3) multi-channel implantation is not possible.

### SUMMARY

In order to solve the above existing technical problems, defects, and technical requirements that cannot be achieved, the present disclosure aims to provide a flexible delivery system for a radioactive source. The present disclosure uses a flexible pushing wire to push seeds, or seed strands, or seed strand casings, to realize a high-precision position control and high-precision seed implantation, and at the same time, through a flexible delivery tube in the delivery channel, the system can better adapt to drifting movement of the puncture needle caused by a patient's respiration, heartbeat, or body trembling, etc., to ensure the safety of the patient. A cutting mechanism in the present disclosure can adjust the length of the seed strand casing according to a feature of a tumor and requirements of surgery. In addition, the present disclosure can also realize a multi-channel implantation of seeds, which further improves the implantation efficiency of radioactive sources and solves the technical defects of the existing technology.

In order to achieve the above purposes, the present disclosure proposes a flexible delivery system for a radioactive source comprising a delivery channel, a flexible pushing wire, and a wire driving mechanism. The flexible pushing wire is disposed inside the delivery channel and the delivery channel is configured to guide the flexible pushing wire to move forward and backward. The flexible pushing wire may be disposed in the delivery channel, the delivery channel being configured to guide the flexible pushing wire to move forward and backward, the wire driving mechanism being configured to drive the flexible pushing wire to move reciprocally along the delivery channel, and the flexible pushing wire being configured to push seeds, or seed strands, or seed strand casings, to be delivered to a target position along the delivery channel. The delivery channel may include a flexible delivery tube and a wire output channel, the flexible delivery tube being connected with the wire output channel, and a length of the flexible pushing wire being greater than 600 mm.

The flexible delivery system for a radioactive source of the present disclosure realizes flexible pushing of the radioactive source and selectively docking with a puncture needle by providing the delivery channel, the flexible pushing wire, and the wire driving mechanism, which effectively avoids the problem of easily scratching the patient through a rigid connection. The flexible pushing wire is configured to realize the pushing of the seeds, the seed strands, or the seed strand casings, and to realize high-precision position control and high-precision implantation of seeds to adapt to the drifting movement of the puncture needle caused by the patient's respiration, heartbeat, or body tremor, which ensures the safety of the patient.

In some embodiments, the flexible pushing wire may be a flexible wire with elasticity, the flexible pushing wire may be capable of being bent under an external force and being restored to a straight state after the external force is withdrawn. A material of the flexible pushing wire may be one or a combination of several types, including nickel-titanium alloy, spring steel, and composite materials. The composite material may specifically include a carbon fiber composite material and a glass fiber composite material.

Adopting the flexible pushing wire with a certain degree of elasticity and flexibility to push the seed can ensure safety while stably driving a friction wheel, which is conducive to the transmission of a driving force from a rear end to a front end of the flexible pushing wire, to realize seed pushing over a long distance.

In some embodiments, the wire output channel may include a first flexible section, the first flexible section being a flexible and bendable tube, and a length of the first flexible section may be more than 200 mm and the wire output channel may be a plastic tube or a medical braided tube. A length of the flexible delivery tube may be greater than 600 mm and the flexible delivery tube may be a plastic tube or a medical braided tube.

The flexible pushing wire and the flexible delivery tube have a certain degree of suppleness, which can adapt to the drifting movement of the puncture needle caused by the patient's respiration, heartbeat, or body tremor, etc., to solve the existing technical defects.

In some embodiments, one end of the flexible delivery tube may be connected with the puncture needle or a quick-connection coupler capable of connecting with the puncture needle, and the quick-connection coupler may be connected with the puncture needle through at least one or a combination of thread, latch, or adhesive.

A front end of the puncture needle may be provided with a first sharp portion, the puncture needle may be made of a bendable and deformable material, an elastic modulus of the bendable and deformable material being less than 50 GPa, and the bendable and deformable material including at least one of nickel-titanium alloy, plastic, or a composite material. The composite material may specifically include a carbon fiber composite material and a glass fiber composite material.

The puncture needle connected with the flexible delivery tube in the present disclosure is a flexible puncture needle. The flexible puncture needle may include a flexible needle trocar, the flexible needle trocar being a hollow tubular structure made of a bendable and deformable material. When the flexible needle trocar is punctured into the tissues of a living organism, the flexible needle trocar automatically adapts to and deforms according to a force of the organism's tissues on the flexible needle trocar to avoid scratches. By using the flexible puncture needle, the effect of forcibly straightening the flexible needle trocar can be minimized.

The flexible needle trocar may have an inner diameter in a range of 0.5 mm to 1.5 mm and a wall thickness in a range of 0.01 mm to 0.5 mm, and may be made of a bendable and deformable material, specifically including at least one of plastic, nickel-titanium alloy, silicone, or rubber; an outer diameter of a rigid stylet may be smaller than the inner diameter of the flexible needle trocar, the rigid stylet being made of a material with better rigidity, such as at least one of stainless steel, high-speed steel, or tungsten steel.

In some embodiments, the flexible delivery tube may be provided with an air exhaust structure. The air exhaust structure may be configured to equalize air pressures inside and outside of the flexible delivery tube when the flexible pushing wire reciprocally moves along the flexible delivery tube.

The interior of the flexible delivery tube may be provided with a flexible stylet, the flexible stylet may be clearance-matched with the flexible delivery tube and may be pluggable relative to the flexible delivery tube, and the flexible stylet may be a flexible wire with elasticity, and capable of being bent under an external force and being restored to a straight state after the external force is withdrawn, and a length of the flexible stylet may be greater than 600 mm, the rear end of the flexible stylet may extend backward from a rear end of the flexible delivery tube, and a front end of the flexible stylet may be located within the interior of the puncture needle connected with a front end of the flexible delivery tube.

According to good suppleness, the flexible stylet may be adaptively deformed according to the force of the organism's tissues on the flexible needle trocar to avoid scratches. The flexible stylet may be made of a bending and deformable material, and specifically including at least one of plastic, nickel-titanium alloy, silicone, latex, or rubber. The flexible stylet may be made of nickel-titanium alloy and soft material combination, and a main body of the flexible stylet may be a nickel-titanium alloy wire whose outer diameter is slightly smaller than the inner diameter of the flexible needle trocar, but a diameter-changing section may be disposed at a front end of the nickel-titanium alloy wire, an outer diameter of the diameter-changing section may be smaller than the outer diameter of the main body, so as to realize a lower stiffness and better suppleness. The nickel-titanium alloy wire may be provided with a filler soft sleeve on an outer side of the diameter-changing section, an outer diameter of the filler soft sleeve may be the same as the outer diameter of the main body, so as to realize the consistency of the outer diameter of the flexible stylet in a whole with a low rigidity at the front end and a high rigidity at the rear end of the flexible stylet. A material of the filler soft sleeve may be at least one of plastic, silicone, latex, or rubber, and a length of the diameter-changing section may be in a range of 10 mm to 300 mm.

The flexible delivery tube may be a bendable and flexible tube which is made of at least one of plastic, rubber, latex, or silicone.

In some embodiments, the flexible delivery tube may include an inner tube and an outer tube, the outer tube being sleeved outside the inner tube, the inner tube may be clearance-matched with the outer tube and may be pluggable relative to the outer tube. The inner tube may be configured to be connected with a puncture needle.

In some embodiments, the inner tube and the outer tube are made of a plastic tube or a medical braided tube, and the plastic tube may be made of a polytetrafluoroethylene (PTFE) material.

In some embodiments, a mounting bracket may be provided on the wire driving mechanism, the mounting bracket being provided with a wheel-shaped container with a concave inner surface, the flexible pushing wire being coiled on a concave inner side of the wheel-shaped container, and the wheel-shaped container rotates freely on the mounting bracket, and under an elastic action of the flexible pushing wire, the flexible pushing wire may be automatically coiled inside the concave inner side of the wheel-shaped container.

In some embodiments, the system may further include a radioactive source feeding mechanism, the radioactive source feeding mechanism being configured to provide the seeds, or the seed strands, or the seed strand casings through a magazine. The seeds, or the seed strands, or the seed strand casings may be mounted in a storage slot or a storage hole inside the magazine, and the seeds, or the seed strands, or the seed strand casing may be provided at the front of the flexible pushing wire for feeding through a magazine feeding mechanism mounted on the magazine. The seed strands may include one or more seeds and seed strand casings, two adjacent seeds may be connected by the seed strand casings, and the seed strand casings may be made of a human-biodegradable material. The seeds and seed strand casings may be connected with each other by adhesive or interference fit, or the seed strand casings may be sleeved outside the seeds, the seed strand casings may be made of a human-biodegradable material, and the human-biodegradable material may include at least one of collagen, polymer polymers, gelatin, alginate, or a polyester degradable material.

In some embodiments, the radioactive source feeding mechanism may be a seed magazine or a seed strand magazine, and the wire driving mechanism may include the seed magazine or the seed strand magazine configured to store radioactive seeds and a seed pushing driving mechanism configured to drive the flexible pushing wire to push the radioactive seeds out of the seed magazine or the seed strand magazine and deliver the radioactive seeds along the delivery channel.

In some embodiments, the system may further include a first motion platform and a first connection portion, one end of the wire output channel and the first connection portion being mounted on opposite sides of the first motion platform, respectively. The first motion platform may be configured to realize a relative movement in space between the one end of the wire output channel and the first connection portion. The first connection portion may be connected with a first connection part, the first connection part being provided with a plurality of connection holes, and the first connection portion being at least one of an adhesive connection portion, a welded connection portion, a threaded connection portion, a snap connection portion, or a lock connection portion. One end of each of the multiple flexible delivery tubes is installed on a different connection hole of the first connection part. The wire output channel may be configured to form a delivery channel for the seeds, or the seed strands, or the seed strand casings by docking with one of the flexible delivery tubes on the first connection part to realize a multi-channel delivery.

In some embodiments, the first motion platform may be configured to control a relative movement in space between the one end of the wire output channel and the first connection portion by: controlling the first connection part to move and the one end of the wire output channel to be stationary; controlling the first connection part to be stationary and the one end of the wire output channel to move; or controlling the first connection part to move and the one end of the wire output channel to move.

In some embodiments, the first motion platform may be configured to realize a relative movement of at least two degrees of freedom between the first connection part and the one end of the wire output channel, and the relative movement includes at least one of:
A. the first connection part is stationary, and the one end of the wire output channel performs a back-and-forth linear movement and a movement within a plane;
B. the first connection part performs a back-and-forth linear movement, and the one end of the wire output channel performs a movement within a plane;
C. the first connection part performs a movement within a plane, and the one end of the wire output channel performs a back-and-forth linear movement; or
D. the first connection part performs a back-and-forth linear movement within a plane, and the one end of the wire output channel is stationary; and
   the movement within a plane is one of the following types: a single rotational movement, a single rotational movement combined with a radial linear movement, a double-joint rotational movement, or a double linear movement along X and Y axes.

In some embodiments, the first motion platform may include a back-and-forth movement module, a rotational movement module, and a radial movement module, and the first motion platform may realize a movement of three degrees of freedom of the one end of the wire output channel in space through a one-direction rotational movement and a two-direction linear movement; or the first motion platform may include the back-and-forth movement module and the rotational movement module, and the first motion platform may realize a movement of the one end of the wire output channel in space through a one-direction rotational movement and a one-direction linear movement; or the first motion platform may include the back-and-forth movement module, a left-and-right movement module, and an up-and-down movement module, and the first motion platform may realize the movement of three degrees of freedom of the one end of the wire output channel in space through a three-direction linear movement; or the first motion platform may be a multi-jointed robotic arm driving the one end of the wire output channel to freely move and to be positioned in a three-dimensional space.

In some embodiments, when the first motion platform includes the back-and-forth movement module and the rotational movement module, the first motion platform may be in a shape of a drum, the wire driving mechanism may be disposed inside the drum, the rotational movement module drives the drum to rotate around an axis of the drum, and the back-and-forth movement module (implantation docking mechanism) may be disposed inside the drum for driving the one end of the wire output channel to move forward and backward.

In some embodiments, the plurality of flexible delivery tubes may be secured to the first connection part through a quick connector, the wire driving mechanism and a docking tube structure may be mounted on the first motion platform, when the wire driving mechanism and the docking tube structure are connected through a flexible connection tube, the flexible connection tube may be a portion of the wire output channel, and an outlet of the docking tube structure may be a terminal end of the wire output channel, the docking tube structure may be inserted into a tapered hole on the quick connector to realize docking, and the docking tube structure may adopt a floating docking tube structure for floating within a range to realize adaptive alignment docking.

In some embodiments, a plurality of conical holes may be distributed on a side of the first connection part, and a plurality of connection holes may be distributed on the other side of the first connection part, each connection hole may directly correspond to each conical hole, the flexible delivery tube may be installed in the connection hole of the first connection part through an quick connector. The connection hole and the quick connector may be connected through a lock structure, a threaded structure, and an interference fit. An end of the wire output channel close to the first connection part may be also connected with a docking tube structure, and the docking tube structure may be provided with a conical docking tube structure, the conical docking tube structure may be capable of cooperating with a centered conical surface on the connection hole. A floating connection mechanism may be provided between the docking tube structure and the first motion platform, or inside the first motion platform, or between the first motion platform and the first connection part. The floating connection mechanism may be configured to cause a relative movement between the docking tube structure and the first motion platform and, or inside the first motion platform, or between the first motion platform and the first connection part when the docking tube structure or the first connection part is subjected to an external force, and the docking tube structure may be automatically restored when not subjected to the external force. The relative movement between the first connection part and the docking tube structure may be realized by a motion platform, so as to realize the switching of a plurality of wire output channels, and the docking tube structure can automatically align with a plurality of input holes on the first connection part. The docking tube structure has a conical surface, which may be adapted to a conical hole on the first connection part, and may be automatically aligned with the conical hole even in the case of a small positioning error.

The floating connection mechanism may include a ball-hinge rod structure, and the floating elastic element may be at least one of elastic blocks, elastic pads, springs, or elastically supported beams.

In view of the shortcomings of the prior art, the present disclosure further proposes a method for using the flexible delivery system for a radioactive source, including:
Step 1.1: connecting a plurality of flexible delivery tubes with a connection part through a quick connector, respectively, wherein a front end of each of the flexible delivery tubes is connected with one of the hollow puncture needles inserted into a target body;
Step 1.2: positioning, through a movement of a first motion platform, a docking tube structure at a terminal end of a wire output channel and different quick connectors on the first connection part, and docking, through a back-and-forth movement of the first motion platform, the docking tube structure at the terminal end of the wire output channel with the different quick connectors on the first connection part; and
Step 1.3: driving, by a wire driving mechanism, a flexible pushing wire to push out seeds, or seed strands, or seed strand casings disposed in front of the flexible pushing wire by a radioactive source feeding mechanism to a target position through a flexible delivery tube and implanting the seeds, or the seed strands, or the seed strand casings into the target body through the plurality of hollow puncture needles.

The present disclosure has the following beneficial effects compared with the prior art.

1. The present disclosure employs the flexible pushing wire to realize the pushing of the seeds, or the seed strands, or the seed strand casings, and at the same time, a position detection component is disposed inside the wire driving mechanism, so that an actual position of the flexible pushing wire can be measured in real-time, thus realizing high-precision position control and high-precision seed implantation. In addition, the flexible pushing wire and the flexible delivery tube have a certain degree of suppleness, which can adapt to the drifting movement of the puncture needle caused by the patient's respiration, heartbeat, body tremor, etc., thereby ensuring the safety of the patient and solving the technical defects of the existing technology.

2. The present disclosure can realize a multi-channel delivery by setting the first motion platform and the first connection part, with one end of a plurality of flexible delivery tubes being mounted on the first connection part; one end of the wire output channel or one end of a mixing wire output channel being mounted on the first motion platform. The first motion platform is configured to realize a relative movement in space between the one end of the wire output channel or the one end of the mixing wire output channel and the first connection part, so that the wire output channel or the mixing wire output channel is docked with each flexible delivery tubes on the first connection part to form a delivery channel for the seed or the seed strand, thereby realizing the multi-channel delivery.

### BRIEF DESCRIPTION OF THE DRAWINGS

In order to more clearly illustrate the technical solutions in the specific embodiments or prior art of the present disclosure, the accompanying drawings that need to be used in the descriptions of the specific embodiments or prior art will be briefly described below, and it will be obvious that the following description of the accompanying drawings is the embodiments of the present disclosure, and for the ordinary skilled person in the field, in the premise of no creative labor, other drawings based on the drawings can also be obtained.

FIG. 1 is a schematic diagram illustrating an overall structure of a wire driving mechanism according to some embodiments of the present disclosure;

FIG. 2 is a schematic diagram of an overall structure of the wire driving mechanism according to some embodiments of the present disclosure;

FIG. 3 is a schematic diagram illustrating an overall cross-sectional structure of the wire driving mechanism according to some embodiments of the present disclosure;

FIG. 4 is a schematic diagram illustrating an overall structure of a wire driving mechanism according to some embodiments of the present disclosure;

FIG. 5 is a schematic diagram illustrating an overall structure of a wire driving mechanism according to some embodiments of the present disclosure;

FIG. 6 is a schematic diagram illustrating an exemplary structure of a radioactive source feeding mechanism according to some embodiments of the present disclosure;

FIG. 7 is a schematic diagram illustrating an enlarged view of a partial structure of a radioactive source feeding mechanism according to some embodiments of the present disclosure;

FIG. 8 is a schematic diagram illustrating an exemplary structure of the radioactive source feeding mechanism according to some embodiments of the present disclosure;

FIG. 9 is a schematic diagram illustrating an exemplary connection structure between a seed and a seed strand casing according to some embodiments of the present disclosure;

FIG. 10 is a schematic diagram illustrating an exemplary structure of a wire driving mechanism according to some embodiments of the present disclosure;

FIG. 11 is a schematic diagram illustrating an enlarged view of a partial structure of the wire driving mechanism according to some embodiments of the present disclosure;

FIG. 12 is a schematic diagram illustrating an overall structure of a flexible delivery system for a radioactive source according to some embodiments of the present disclosure;

FIG. 13 is a schematic diagram illustrating a structure of the flexible delivery system for a radioactive source according to some embodiments of the present disclosure;

FIG. 14 is a schematic diagram illustrating an exemplary structure 1 of embodiment 5 of the present disclosure;

FIG. 15 is a schematic diagram illustrating an exemplary structure of a magazine base and a magazine of embodiment 5 of the present disclosure;

FIG. 16 is a schematic diagram illustrating embodiment 5 when a door of device housing is opened of the present disclosure;

FIG. 17 is a schematic diagram illustrating an exemplary structure of a first motion platform of embodiment 5 of the present disclosure;

FIG. 18 is a schematic diagram illustrating an exemplary structure of the first connection part of embodiment 5 of the present disclosure;

FIG. 19 is an internal schematic diagram illustrating a first connection part according to some embodiments of the present disclosure;

FIG. 20 is a schematic diagram illustrating an exemplary structure of a needle pulling driver of embodiment 5 of the present disclosure;

FIG. 21 is a schematic diagram illustrating an enlarged view of position L in FIG. 20 of the present disclosure;

FIG. 22 is a schematic diagram illustrating a first scheme of a synchronized connector locking mechanism and an synchronized stylet locking mechanism of embodiment 5 of the present disclosure;

FIG. 23 is another schematic diagram illustrating a second scheme of the synchronized connector locking mechanism and the synchronized stylet locking mechanism of embodiment 5 of the present disclosure;

FIG. 24 is a schematic diagram illustrating seed implantation into a target body of embodiment 5 of the present disclosure;

FIG. 25 is a schematic diagram illustrating a wheeled storage mechanism of embodiment 5 of the present disclosure;

FIG. 26 is a schematic diagram illustrating an exemplary structure of embodiment 6;

FIG. 27 is a schematic diagram illustrating a docking tube structure and the needle pulling driver of embodiment 4 of the present disclosure;

FIG. 28 is a schematic diagram illustrating an exemplary structure of the docking tube structure of embodiment 6;

FIG. 29 is another schematic diagram illustrating an exemplary structure of the docking tube structure of embodiment 6 of the present disclosure;

FIG. 30 is a schematic diagram illustrating an exemplary cooperation structure of a first link, a second link, and a link seat of embodiment 6 of the present disclosure;

FIG. 31 is a schematic diagram illustrating an exemplary structure of an alternative embodiment of the docking tube structure of embodiment 6 of the present disclosure; and

FIG. 32 is another schematic diagram illustrating an exemplary structure of an alternative embodiment of the docking tube structure of embodiment 6 of the present disclosure.

### DETAILED DESCRIPTION

The technical solutions in the embodiments of the present disclosure will be clearly and completely described below in conjunction with the accompanying drawings in the embodiments of the present disclosure, and it is clear that the embodiments described are only a portion of the embodiments of the present disclosure, not all of the embodiments. Based on the embodiments in the present disclosure, all other embodiments obtained by a person of ordinary skill in the art without making creative labor fall within the scope of protection of the present disclosure.

### Embodiment 1

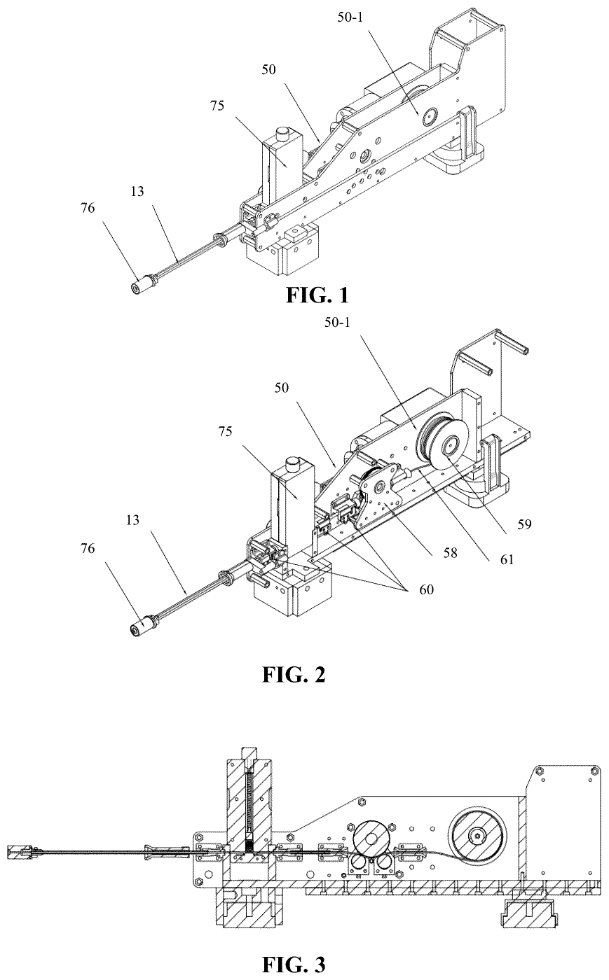

As shown in FIG. 1 to FIG. 5, for drawbacks existing in the prior art, the present disclosure proposes a flexible delivery system for a radioactive source including a wire output channel 13, a flexible pushing wire 61, and a wire driving mechanism 50. The flexible pushing wire 61 is disposed in the wire output channel 13, the wire output channel 13 is configured to guide the flexible pushing wire 61 to move forward and backward, the wire driving mechanism 50 is configured to drive the flexible pushing wire 61 to move reciprocally along the wire output channel 13, and the flexible pushing wire 61 is configured to push seeds, or seed strands, or seed strand casings to be delivered along the wire output channel 13 to a target position.

The seed strand of the present disclosure is a strip containing a radioactive material, the seed strand includes a plurality of seeds and a seed strand casing for connecting the seeds so that the seeds are connected with each other sequentially to form a chain-like structure.

The seed strand casing is a tube configured to encapsulate the plurality of seeds, the tube may be in a semi-enclosed structure, and two ends and one side surface of the tube may be open to enable the seeds to be embedded into the seed strand casing from one end or one side surface of the seed strand casing through a seed embedding mechanism, thereby forming a complete seed strand. The tube is made of a human-biodegradable material including at least one of collagen, polymer, gelatin, alginate, or a polyester-biodegradable material.

The flexible pushing wire 61 is a flexible wire with elasticity, and the flexible pushing wire 61 may be bent under an external force and restored to a straight state after the external force is withdrawn. A material of the flexible pushing wire 61 may include one or a combination of several types, including nickel-titanium alloy, spring steel, and composite materials. The composite material specifically includes a carbon fiber composite material and a glass fiber composite material. A length of the flexible pushing wire 61 is greater than 600 mm.

The wire output channel 13 may include a flexible delivery tube and a wire output channel. The flexible delivery tube and the wire output channel are connected with each other. The wire output channel may include a first flexible section, the first flexible section being a flexible and bendable tube, and a length of the first flexible section is greater than 200 mm and is made of a plastic tube or medical braided tube. The wire output channel may include a second flexible section, the second flexible section being a flexible and bendable tube. A length of the flexible delivery tube is greater than 600 mm and is made of a plastic tube or a medical braided tube.

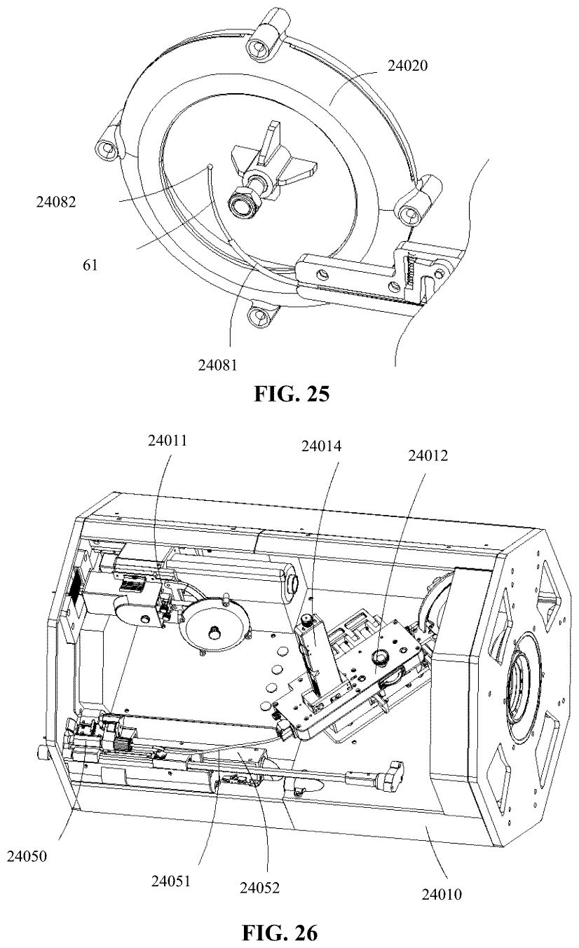

The wire driving mechanism 50 may be provided with a storage mechanism, and the storage mechanism in this embodiment adopts a container 59, as shown in FIG. 25, the container 59 adopts a wheel-shaped container 24020 with a concave inner surface, the wheel-shaped container 24020 may rotate freely around its own axis, and edges of the wheel-shaped container 24020 is a concave structure, a storage region forms at the concave structure. At least one side surface of the wheel-shaped container 24020 is disposed with an opening, and the flexible pushing wire 61 may extend into the opening on the side surface of the wheel-shaped container 24020 guided by a storage guiding tube 24081, and under the elastic action of the flexible pushing wire 61, the flexible pushing wire is automatically coiled inside the concave inner side of the wheel-shaped container, and the wheel-shaped container 24020 rotates as the flexible pushing wire 61 moves forward and backward to realize automatic storage.

The purpose of this embodiment is to provide a flexible delivery system for a radioactive source that connects the wire driving mechanism 50 and the puncture needle 18 through the delivery channel, and the present disclosure employs the flexible pushing wire to push the seeds, or the seed strands, or the seed strand casings, and the seed strand or the seed strand casing of a target length is cut off by a cutting mechanism, so as to realize feeding of radioactive sources. At the same time, there is a position detection component provided inside the wire driving mechanism, which can measure an actual position of the flexible pushing wire in real-time, to realize high-precision position control and high-precision implantation of seeds. In addition, the flexible pushing wire and the flexible delivery tube have a certain degree of suppleness, which can accommodate the drifting movement of the puncture needle 18 caused by the patient's respiration, heartbeat, etc., thereby ensuring the safety of the patient.

The wire driving mechanism provided in this embodiment includes a power component 68, a transmission component 69, a guiding component 70, a mounting bracket 50-1, and a position detection component 60, the transmission component, the guiding component, and the position detection component being all mounted on the mounting bracket 50-1. The power component 68 is configured to power a movement of the flexible pushing wire. The transmission component 69 is configured to transmit a power output from the power component 68 to the flexible pushing wire 61. The guiding component 70 is configured to guide the flexible pushing wire 61. The position detection component 60 is configured to measure a position of the flexible pushing wire 61 relative to guiding component 70 or/and the mounting bracket 50-1. A resistance measuring element is provided within the transmission component 69 or the power component 68, including at least one of force sensors, torque sensors, and current sensors. The resistance measuring element is configured to measure a propulsive resistance subjected by the flexible pushing wire 61.

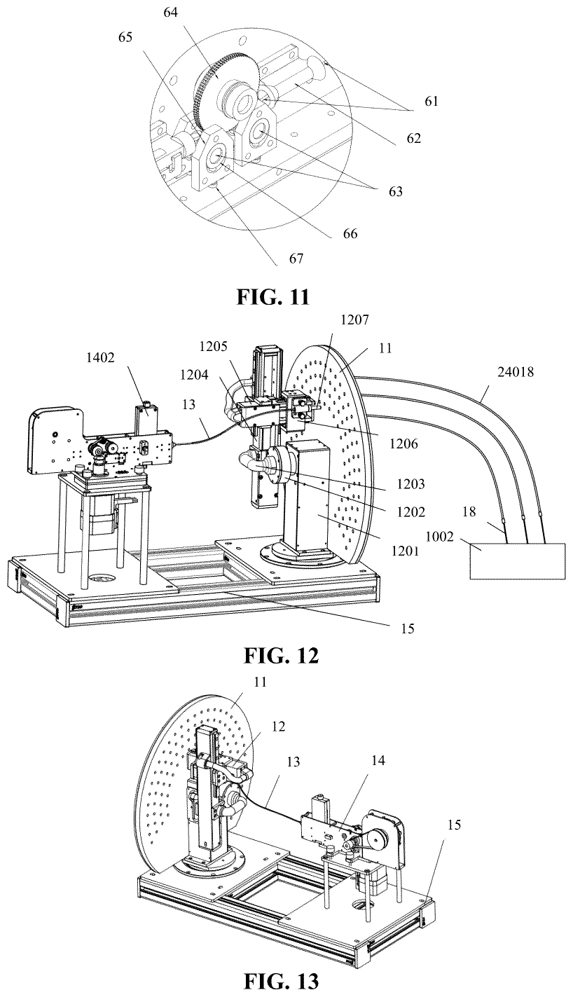

As shown in FIG. 5, FIG. 10, and FIG. 11, the power component 68 is in a form of a first motor 51 or a form of a combination of the first motor 51 and a speed-reducer 52. The speed-reducer 52 is fixed to the mounting bracket 50-1 through a first fixation plate 56, and the power component or the transmission component is connected with an internal angle sensor, which can measure a theoretical displacement of the flexible pushing wire or the seed strand driven by the power component. The transmission component 69 includes at least one second friction assembly 63 and at least one first friction assembly 64, one end of the first friction assembly 64 and/or the second friction assembly 63 is drive-connected with an output shaft of the power component 68.

As shown in FIG. 5, the transmission component 69 includes a bevel gear assembly 53 or/and a spur gear assembly.

As shown in FIG. 10 and FIG. 11, the transmission component 69 also include the second friction assembly 63 and the first friction assembly 64. One end of the first friction assembly 64 and/or the second friction assembly 63 is drive-connected with the output shaft of the power component 68, and the transmission component 69 is disposed between a third fixation plate 58 and a fixation bracket. The second friction assembly 63 is supported on the mounting bracket through a set of position-limiting bases 65, and a support ring 66 is disposed inside each of the position-limiting bases 65, and the support ring 66 supports the second friction assembly 63.

At least one second friction assembly 63 and at least one first friction assembly 64 may be provided.

The flexible pushing wire 61 may pass between the second friction assembly 63 and the first friction assembly 64. The flexible pushing wire 61 is in contact with one side surface of the second friction assembly 63, and the flexible pushing wire 61 is in contact with one side surface of the first friction assembly 64, such that the first friction assembly 64, when rotates, may drive the flexible pushing wire 61 to move forward or backward along the guide component 70.

In some embodiments, the first friction assembly 64 is an active friction wheel or an active friction belt, and the second friction assembly 63 is a passive friction wheel or a passive friction belt or an active friction wheel or an active friction belt.

In some embodiments, the first friction assembly 64 is a passive friction wheel or a passive friction belt, and the second friction assembly 63 is an active friction wheel or an active friction belt.

As shown in FIG. 10, the guiding component 70 may include a guiding base 62 and a guiding tube base 71. The guiding base 62 is mounted on the mounting bracket 50-1, and the position detection component 60 is provided on the guiding component 70. The flexible pushing wire 61 is a bendable and flexible wire. The flexible pushing wire has a certain degree of elasticity, and may return to a straight state when an external force is withdrawn, and a specific material of the flexible pushing wire may include one or a combination of several types, including nickel-titanium alloy, spring steel, and composite materials, and the composite materials specifically includes a carbon fiber composite material and a glass fiber composite material.

A measuring wheel is directly connected with an encoder 55 or is driven to rotate the encoder 55 by the other transmission component. The flexible pushing wire 61 is in contact with one side surface of the measuring wheel, when the flexible pushing wire 61 moves forward or backward along the guide component 70, the flexible pushing wire 61 will drive the measuring wheel and encoder 55 to rotate. The encoder 55 is mounted on the mounting bracket 50-1 by a second fixation plate 57.

A material of the first friction assembly 64 and second friction assembly 63 includes at least one of metal, plastic, ceramic, silicone, or rubber.

The first friction assembly 64 may include at least one friction wheel, a surface of the friction wheel is provided with a transverse slip-resistant slot, the transverse slip-resistant slot has a width in a range of 0.1 mm to 1 mm, and an angle between a direction of the transverse slip-resistant slot and a direction of the flexible pushing wire 61 is greater than 60 degrees.

The first friction assembly 64 and second friction assembly 63 are both provided with an annular slot adapted to the flexible pushing wire 61, preventing the flexible pushing wire 61 from detaching from the friction wheel.

As shown in FIG. 2, the mounting bracket 50-1 is provided with one or more position detection components 60. Each of the one or more position detection components 60 includes a travel switch, or a position encoder, or a displacement sensor, and the position detection component 60 is configured to measure an actual position of the flexible pushing wire 61 or/and the seed strand.

The first motor 51 is configured to drive the active friction wheel to rotate through the bevel gear set 53, the active friction wheel drives the flexible pushing wire 61 to move, and the passive friction wheel follows the rotation and drives the encoder 55 through the spur gear 54. The encoder 55 may measure a displacement length of the flexible pushing wire 61 according to a rotation angle, the guide part 70 may guide the flexible pushing wire 61 to move along the wire output channel, and when the flexible pushing wire 61 passes the travel switch, the flexible pushing wire 61 may contact with a conductive contact in the travel switch to generate a zero position signal. Then, when the flexible pushing wire 61 moves along the wire output channel, the encoder can measure the actual position of the flexible pushing wire 61.

By using the measuring wheel, and the encoder to measure an actual distance, high-precision position control of the flexible pushing wire or the seed strand are realized, a function of implanting the seed or the seed strand can be realized. According to the difference between the nominal displacement amount driven by the motor and the actual displacement amount measured by the encoder, various conditions during a seed implantation process may be determined, such as needle trocar clogging, tube clogging, friction wire driving mechanism slippage, whether a magazine is empty, etc.

### Embodiment 2

A flexible delivery system for a radioactive source, where the parts of this embodiment that are identical to those in Embodiment 1 will not be described again. The differences are as follows:

The flexible delivery system may further include that: one end of a flexible delivery tube is connected with the puncture needle 18 or is provided with a quick-connection coupler 76 capable of connecting with the puncture needle 18, and the quick-connection coupler 76 is connected with the puncture needle 18 through at least one or a combination of thread, latch, or adhesive. The flexible wire output channel 13 includes the wire output channel 13 and the flexible delivery tube 24018.

In some embodiments, since a plurality of seeds usually need to be implanted in a puncture path in which the puncture needle 18 is located, after completing an implantation of one seed, it is necessary to use the flexible delivery tube to pull a needle trocar upwardly by a small section of displacement. Then, through the flexible wire output channel 13, implant another one seed, and the above operation may be repeated several times until all seeds in the puncture path have been implanted.

A front end of the puncture needle is provided with a first sharp portion, specifically, one end of the flexible delivery tube is connected to the needle trocar of the puncture needle through a needle trocar connector, and a front end of the needle trocar is provided with the first sharp portion. In some embodiments, the puncture needle trocar is made of a bendable and deformable material, and an elastic modulus of the bendable and deformable material is less than 50 GPa. The material specifically includes at least one of nickel-titanium alloy, plastic, or a composite material, and the composite material specifically includes a carbon fiber composite material and a glass fiber composite material.

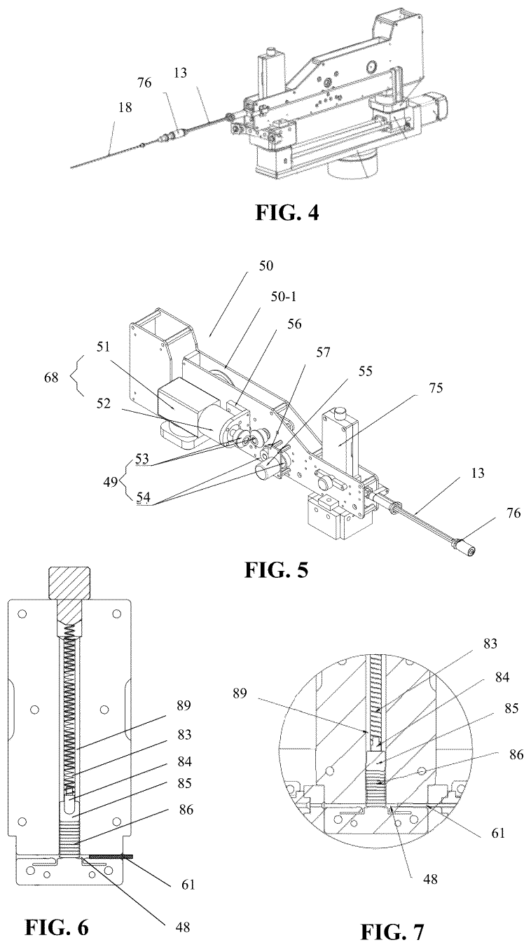

As shown in FIG. 4, the puncture needle 18 connected to the flexible delivery tube in the present disclosure is a flexible puncture needle. The flexible puncture needle includes a flexible needle trocar, and the flexible needle trocar is a hollow tubular structure made of a bendable and deformable material. When inserted into the tissue of an organism, the flexible needle trocar may automatically adapt and deform according to the force of the tissue on the flexible needle trocar to avoid scratches. Using the flexible puncture needle can reduce a situation of scratches caused by the flexible delivery tube pulling the flexible needle trocar.

The flexible needle trocar has an inner diameter in a range of 0.5 mm to 1.5 mm and a wall thickness in a range of 0.01 mm to 0.5 mm. The flexible needle trocar is made of a bendable and deformable material, which may be specifically made of at least one of plastic, nickel-titanium alloy, silicone, or rubber. During puncture, in order to facilitate an operation of a doctor and adjust a puncture angle, a rigid short stylet needs to be inserted into the flexible needle trocar to improve an overall rigidity of the puncture needle 18. An outer diameter of the rigid short stylet is smaller than the inner diameter of the flexible needle trocar, and the rigid short stylet is made of a material with good rigidity, such as stainless steel, high-speed steel, tungsten steel, or one or more combinations thereof, and an elastic modulus of the material with good rigidity is greater than 200 GPa.

The flexible delivery tube is provided with an air exhaust structure, and the air exhaust structure may be configured to equalize air pressures inside and outside of the flexible delivery tube when the flexible pushing wire 61 moves reciprocally along the flexible delivery tube. The flexible delivery tube is provided with a flexible stylet, and the flexible stylet is clearance-matched with the flexible delivery tube and is pluggable relative to the flexible delivery tube. The flexible stylet is a flexible wire with elasticity and capable of being bent under an external force and being restored to a straight state after the external force is withdrawn. A length of the flexible stylet is greater than 600 mm, a portion of a rear end of the flexible stylet extends backward from a rear end of the flexible delivery tube, and a front end of the flexible stylet is extended into the puncture needle connected with a front end of the flexible delivery tube.

The flexible stylet has good suppleness, and thus may be adaptively deformed according to the force of the organism's tissues on the flexible needle trocar, avoiding scratches to the organism's tissues. The flexible stylet is made of a bendable and deformable material, specifically including plastic, nickel-titanium alloy, silicone, latex, rubber, or the like, or any combination thereof. Preferably, the flexible stylet is made of a combination of the nickel-titanium alloy and a soft rubber material, and a main body of the flexible stylet is a nickel-titanium alloy wire whose outer diameter is slightly smaller than the inner diameter of the flexible needle trocar.

### Embodiment 3

A flexible delivery system for a radioactive source, where the parts of this embodiment that are identical to those in Embodiment 1 will not be described again. The differences are as follows:

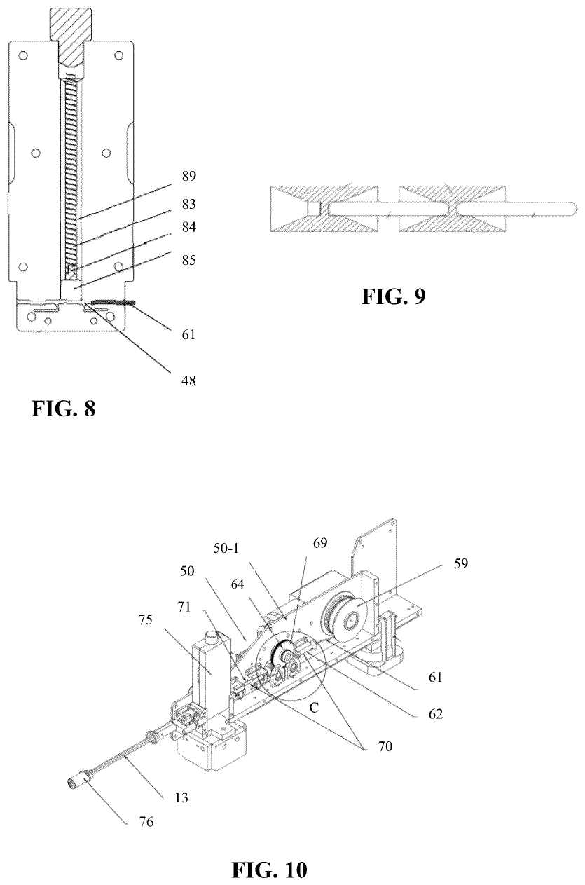

As shown in FIG. 1 to FIG. 10, the flexible delivery system further includes a radioactive source feeding mechanism. The radioactive source feeding mechanism is disposed on the wire driving mechanism 50 or on a wire driving mechanism, the wire output channel 13 configured for guide a movement of the flexible pushing wire 61 is disposed on the wire driving mechanism 50, and the wire driving mechanism is configured to drive the flexible pushing wire 61 to move along the wire output channel 13 reciprocally, and the radioactive source feeding mechanism is configured to provide seeds, or seed strands, or seed strand casings through a magazine. The seeds, or the seed strands, or the seed strand casings are mounted in a storage slot or a storage hole inside a magazine, and the seeds, or the seed strands, or the seed strand casings are disposed at the front of the flexible pushing wire 61 for feeding through a magazine feeding mechanism mounted on the magazine. The flexible pushing wire 61 may deliver the seeds, or the seed strands, or the seed strand casings along the wire output channel 13 to a target position.

The magazine adopts a seed magazine or a seed strand magazine, and the seed magazine or the seed strand magazine adopts a linear magazine, or a drum magazine, or a revolver magazine.

The seed magazine or the seed strand magazine is disposed at any position of a wire output channel, and a seed output channel within the seed magazine or the seed strand magazine is connected with the wire output channel, and a pushing wire may push a seed or a seed strand located in the seed output channel to move.

As shown in FIG. 6 to FIG. 8, the magazine 75 includes a bin body, a seed-pushing device, and a guiding slot 89 provided inside the bin body. The seed-pushing device includes a pushing piece 85, a guiding block 84, and a spring 83. The guiding block 84 is slidably provided in the guiding slot 89, the spring 83 is pressed against the guiding block 84, and the pushing piece 85 is provided inside the bin body below the guiding block and is in contact with the seeds 86 or the seed strands. A seed output channel 48 is disposed at a bottom of the bin body, and the pushing piece 85 pushes the seeds or the seed strands into the seed output channel 48 constantly.

The magazine 75 may be disposed directly at the wire driving mechanism, and the seed output channel 48 within the magazine 75 may be connected with the wire output channel, and the flexible pushing wire 61 may push the seed or seed strand in the seed output channel 48 to move out of the magazine 75.

In some embodiments, a flexible connection tube is disposed at a front end of the wire driving mechanism, and the flexible connection tube extends by a certain distance and is connected with a magazine base. The magazine 75 is mounted on the magazine base. The flexible pushing wire may move through the flexible connection tube to the magazine base and push the seed or seed strand in the seed output channel 48 to move out of the magazine 75. The magazine base may be mounted on a first motion platform and driven by the first motion platform to dock with different delivery tubes to realize a multi-channel implantation. The magazine 75 may also be configured to feed the seed strand, and it is only necessary to widen a storage slot used for storing seeds and widen the pushing piece 85 for pushing the seeds to store the seed strand.

### Embodiment 4

A flexible delivery system for a radioactive source, where the parts of this embodiment that are identical to those in Embodiment 1 will not be described again. The differences are as follows:

The flexible delivery system may further include a first motion platform and a first connection portion, one end of the wire output channel and the first connection portion are mounted on opposite sides of the first motion platform, respectively. The first motion platform is configured to realize a relative movement in space between the one end of the wire output channel and the first connection portion. The first connection portion is connected with a first connection part, the first connection part is provided with a plurality of connection holes, and the first connection portion is at least one of an adhesive connection portion, a welded connection portion, a threaded connection portion, a snap connection portion, or a lock connection portion. One end of each of a plurality of flexible delivery tubes 24018 is mounted on the first connection part, respectively, and the other end of each of the plurality of flexible delivery tubes 24018 is connected with puncture needles 18, respectively. The first motion platform is configured to realize a relative movement in space between the one end of the wire output channel and the first connection part, and the wire output channel is configured to form a delivery tube for the seeds, or the seed strands, or the seed strand casings by connecting with any one of the flexible delivery tubes on the first connection part to realize a multi-channel implantation. The first motion platform is configured to control the relative movement in space between the one end of the wire output channel and the first connection part as follows: controlling the first connection part to move and the one end of the wire output channel to be stationary, controlling the first connection part to be stationary and the one end of the wire output channel to move, or controlling the first connection part to move and the one end of the wire output channel to move.

The first motion platform is configured to realize a relative movement of at least two degrees of freedom between the first connection part and the one end of the wire output channel, and the relative movement includes at least one of: A. the first connection part is stationary, and the one end of the wire output channel performs a back-and-forth linear movement and a movement within a plane; B. the first connection part performs a back-and-forth linear movement, and the one end of the wire output channel performs a movement within a plane; C. the first connection part performs a movement within a plane, and the one end of the wire output channel performs a back-and-forth linear movement; or D. the first connection part performs a back-and-forth linear movement within a plane, and the one end of the wire output channel is stationary; and the movement within a plane is one of the following types: a single rotational movement, a single rotational movement combined with a radial linear movement, a double-joint rotational movement, or a double linear movement along X and Y axes.

There are a plurality of connection holes distributed on the first connection part. One end of the flexible delivery tube is installed within the connection hole on the first connection part through a quick connector. The connection hole and the quick connector are connected through a lock structure, a threaded structure, and an interference fit. An end of the wire output channel close to the first connection part is also connected with a docking tube structure. The docking tube structure includes a tapered docking head, and the tapered docking head cooperates with the centering taper surface of the connection hole or the centering taper surface of the quick connector. A floating connection mechanism is disposed between the tapered docking head and the first motion platform, or within the first motion platform, or between the first motion platform and the first connection part. Preferably, the floating connection mechanism is a part of the docking tube structure, and the floating connection mechanism is disposed between the tapered docking head and the base of the docking tube structure. The base of the docking tube structure is mounted on the first motion platform, and the floating connection mechanism may enable the tapered docking head to move relatively with respect to the base of the docking tube structure when the docking head is subjected to an external force. So, when the docking head is inserted into the connection hole of the first connection part, it may be automatically aligned under the guidance of the centering tapered surface to eliminate a positioning error of the first motion platform, and after the external force is withdrawn, the docking tube structure may be automatically reset.

The relative movement between the first connection part and the docking tube structure is realized by the first motion platform, so as to realize the switching of a plurality of flexible delivery tubes, and the docking head may be automatically aligned when it is docked to a plurality of the flexible delivery tubes on the first connection part, and the docking head has a tapered surface, which may be adapted to the centering taper surface of the connection hole or the centering taper surface of the quick connector, and may be automatically compressed and aligned even with a small positioning error.

The floating connection mechanism is disposed between the first motion platform and the first connection part, and a floating position is between the quick connector and the first motion platform, or within the first motion platform, or between the first connection part and the first motion platform.

As shown in FIG. 12 and FIG. 13, the flexible delivery system for a radioactive source is provided, including a connection part 11, a motion platform 12, a wire output channel 13, a wire driving mechanism 14, and a base 15. The motion platform 12 and the wire driving mechanism 14 are mounted on the base 15, one end of the wire output channel 13 is mounted on the motion platform 12, and the other end of the wire output channel 13 is connected with the wire driving mechanism 14. The motion platform 12 is configured to realize a movement in space of one end of the wire output channel 13, and to realize the docking between the wire output channel 13 and the first connection part 11, and a flexible pushing wire is disposed within the wire output channel 13. The wire driving mechanism 14 includes a magazine 1402 for feeding radioactive sources, and a pushing driving mechanism for driving the flexible pushing wire to push the radioactive sources out of the magazine 1402 and deliver the radioactive sources along the wire output channel 13.

The motion platform 12 realizes a movement of three degrees of freedom of a front end of the wire output channel 13 in space through a one-direction rotational movement and a two-direction linear movement. The wire output channel 13 ensures its own flexibility while realizing a radioactive source delivery and guiding function, thus enhancing the adaptability of the seed delivery channel. The wire driving mechanism 14 provides radioactive sources and a power to deliver the radioactive sources to realize an implantation.

The connection part 11 and the motion platform 12 are mounted on the base 15 through a swivel joint to providing a degree of rotational freedom to adjust a direction of the first connection part 11, and the wire driving mechanism 14 is fixedly connected to the base 15. The first connection part 11 is configured to connect the wire output channel 13 and connect the puncture needle 18 through the plurality of flexible delivery tubes 24018, the puncture needle 18 is inserted into a target object 1002 so that radioactive seeds are guided and delivered through the wire output channel 13 and the first connection part 11 to the puncture needle 18 until they are implanted into the target object 1002. The motion platform 12 is a platform capable of free movement, including three parts: a back-and-forth movement module 1205, a rotational movement module 1201, and a radial movement module 1204, which realizes movements of three degrees of freedom.

A docking tube structure 1207 is mounted on the movement platform 1206 of back-and-forth movement module 1205, and the docking tube structure 1207 includes a docking head and a floating connecting mechanism. The docking head is mounted on the movement platform 1206 via the floating connecting mechanism which enables the docking head to float within a range.

### Embodiment 5

A flexible delivery system for a radioactive source is provided, the parts of this embodiment that have the same structure as those of Examples 1 to 4 are not described in detail, and differences are as follows.

The flexible delivery tube includes an inner tube and an outer tube, the outer tube is sleeved outside the inner tube, the inner tube is clearance-matched with the outer tube and is pluggable relative to the outer tube. The inner tube is configured to be connected with a puncture needle, and the inner tube and the outer tube are made of a plastic tube or a medical braided tube.

Preferably, the plastic tube is made of a PTFE material.

A first motion platform includes a back-and-forth movement module and a rotational movement module, and the first motion platform realizes the movement of one end of the wire output channel in the space through a one-direction rotational movement and a one-direction linear movement.

When the first motion platform is disposed inside a main body housing and includes the back-and-forth movement module and the rotational movement module, a front side of the main body housing is a connection part panel (first connection portion). The first connection part panel is mounted with a connection part, the connect part is provided with a plurality of connection holes on a rotational track of the first motion platform. Each connection hole may be connected to a delivery tube, and the other end of the delivery tube is connected with a puncture needle. The motion platform is in a shape of a drum, and a wire driving mechanism is disposed in the drum. The rotational movement module drives the drum to rotate around an axis of the drum, and the back-and-forth movement module is disposed in the drum for driving one end of the wire output channel to move forward and backward.

A door is disposed on a left and/or the right side of the main body housing, and a combination of a wire driving mechanism, a stylet pulling mechanism, and a magazine base, or a combination of a wire driving mechanism, a stylet pulling mechanism, and a docking tube structure may be mounted on the first motion platform by opening the door. The stylet pulling mechanism, the magazine base, and the docking tube are mounted perpendicular to the first connection part panel, and a magazine is mounted on the magazine base or mounted on the wire driving mechanism.

When the wire driving mechanism and the magazine base are installed on the first motion platform, the wire driving mechanism and the magazine base are connected by a flexible connection tube, and at this time, the flexible connection tube is a part of the wire output channel, and an outlet of the magazine base is a docking head, which is a terminal end of the wire output channel.

When the wire driving mechanism and the docking tube structure are mounted on the first motion platform, the wire driving mechanism and the docking tube structure are connected by the flexible connection tube, and at this time, the flexible connection tube is a part of the wire output channel, an outlet of the docking tube structure is a terminal end of the wire output channel, and the docking tube structure adopts a floating docking tube structure which can float within a certain range.

The delivery tube is quickly mounted to the connection hole of the first connection part by means of a quick connector, and the first connection part is provided with a synchronized connector locking mechanism, which may lock and position the quick connector of each delivery tube simultaneously.

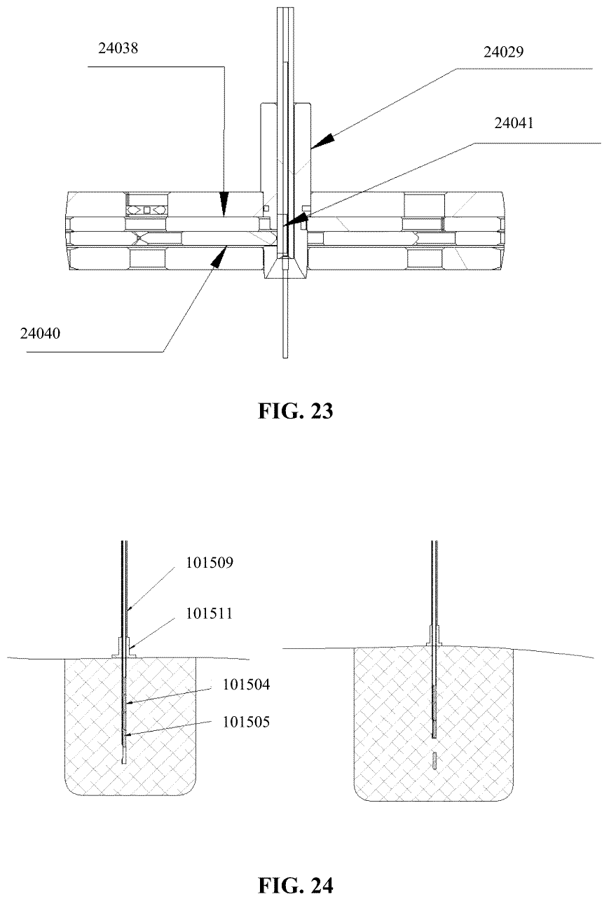

As shown in FIG. 23, the synchronized connector locking mechanism includes a connector clamping plate 24038 disposed within the first connection part, and connector clamping portions are arranged in a circular array on the connector clamping plate 24038. In an unlocked state, all connector clamping portions are staggered with the connection holes, and the quick connector 24029 of the delivery tube can be inserted into the connection holes. When locking, the connector clamping plate 24038 is driven by a clamping driving mechanism to rotate around a center axis, and the connector clamping portions extend into the connection holes. A side of the quick connector 24029 of the delivery tube is provided with a slot, and the quick connector 24029 of the delivery tube is fixed on the first connection part when the connector clamping plate 24038 is rotary to clamp into the slot.

The clamping driving mechanism is electrically or manually actuated and drives the connector clamping plate to rotate around the central axis through cams, gears, or belt drives.

A stylet is disposed inside the delivery tube, extends all the way along the delivery tube, and fills up a space inside the puncture needle connected at a front end of the delivery tube, so as to avoid the blood from pouring into the puncture needle to coagulate and form a clogging. The first connection part is also provided with a synchronized stylet locking mechanism, which may lock and position the stylet inside each delivery tube simultaneously.

As shown in FIG. 23, the synchronized stylet locking mechanism includes a stylet clamping plate 24040 disposed inside the first connection part, stylet clamping portions are arranged in a circular array on the stylet clamping plate 24040. In the unlocked state, all stylet clamping portions are staggered from the connection holes, and the quick connector 24029 of the delivery tube can be inserted into the connection holes. When locking, the stylet clamping plate 240 rotates around a central axis driven by a stylet driving mechanism, and each of the stylet clamping portions inserts into the interior of each of the connection holes. An opening connected to an elastic body 24041 inside the quick connector 24029 is provided on the side of the quick connector 24029 of the delivery tube, and the elastic body 24041 adopts one or a combination of an elastomer, a rubber tube, or a TPU tube. The elastic body 24041 is adjacent to the stylet, and the stylet clamping plate 240 presses against the elastic body 24041 during rotation to press down the stylet, thereby realizing the locking.

In some embodiments, the synchronized connector locking mechanism is the synchronized stylet locking mechanism, the connector clamping plate is the stylet clamping plate, and the clamping driving mechanism is the stylet driving mechanism, and when the clamping driving mechanism drives the connector clamping plate to rotate around a center axis by a first angle, the connector clamping plate inserts into the slot of the quick connector of the delivery tube while not holding against the elastic body. The connector clamping plate fixes the quick connector of the delivery tube on the first connection part, which just realizes locking of the quick connector of each delivery tube but not realizes locking of the style inside each delivery tube. When the clamping driving mechanism drives the connector clamping plate to rotate around the center axis by a second angle, the connector clamping plate presses against the elastic body during rotation, and then presses the stylet, which realizes locking of the quick connector and the style inside each delivery tube.

The stylet needs to be pulled out before an implantation, the stylet is a flexible wire with elasticity, which may be bent under an external force and restored to a straight state after the external force is withdrawn. A length of the stylet is greater than 600 mm. A rear end of the stylet extends backwardly by a portion from a rear end of the delivery tube, and the rear end of the stylet is provided with a first stopping step, and when the first stopping step is pressed against the rear end of the delivery tube, a distance between a front end of the stylet and the front end of the puncture needle connected to the front end of the delivery tube is no more than 5 mm.

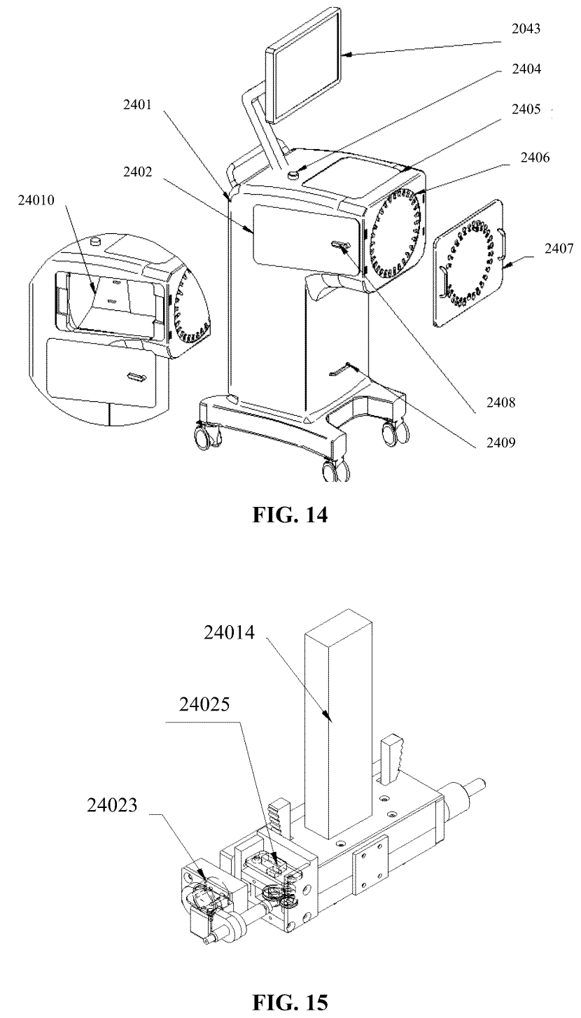

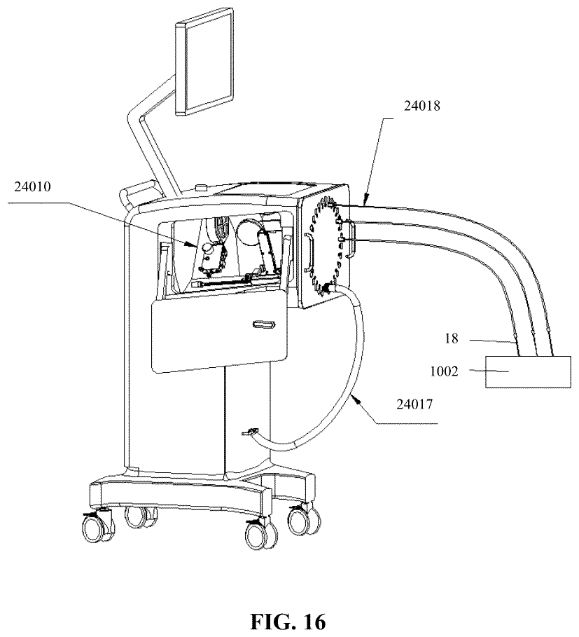

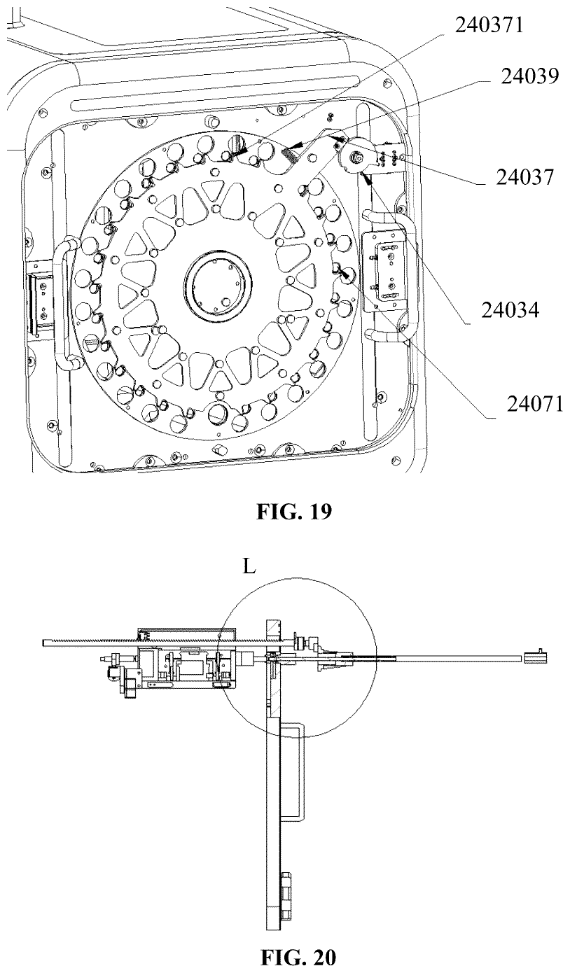

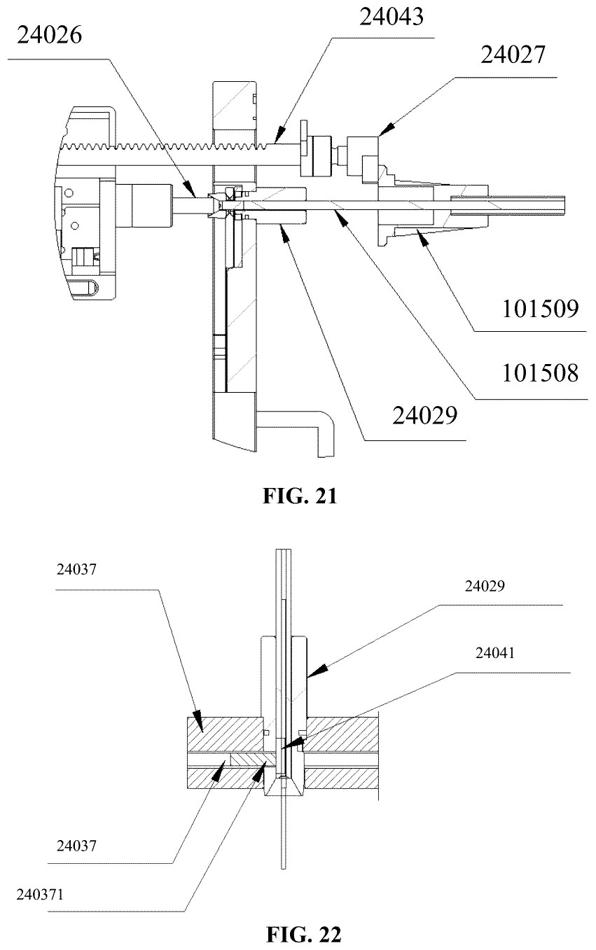

As shown in FIG. 14 to FIG. 25, the first motion platform is the drum, the drum is disposed inside the main body housing, the drum has a one-direction rotational movement around the axis of the drum. There are an implantation docking mechanism and a stylet pulling docking mechanism inside the drum. And the wire driving mechanism, the stylet pulling mechanism, and the magazine base are provided on the drum. The radioactive source feeding mechanism adopts a magazine, the first connection part adopts a docking plate, and a needle pulling driver drives the inner tube or outer tube to make a relative sliding movement between the inner tube or outer tube by direct pushing and pulling.

A main body of a radioactive source implantation mechanism includes a main body housing 2401, a door 2402, a display screen 2403, an emergency stop button 2404, an observation window 2405, a docking plate panel 2406, a docking plate 2407, a door handle 2408, a support column 2409, and a drum 24010.

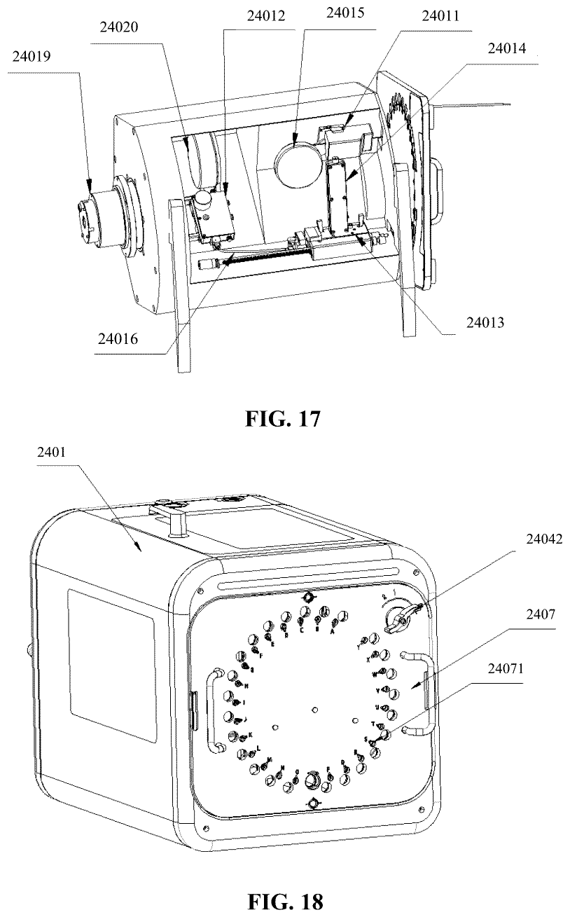

Before the surgery, the door 2402 is in a closed state, the door 2402 is opened by rotate the door handle 2408, the door 2402 is located on the left and/or right side of the main body housing 2401, accessories are installed inside the drum 24010. Specifically, the stylet pulling mechanism 24011, the wire driving mechanism 24012, and the magazine base 24013 are installed in corresponding positions in the drum 24010. The stylet pulling mechanism 24011 and the magazine base 24013 are vertically mounted on the docking plate panel 2406 (referring to FIG. 17). A stylet storage wheel 24015 is disposed at the rear side of the stylet pulling mechanism 24011, and a wire storage wheel 24020 is provided at the rear side of the wire driving mechanism 24012, and the flexible pushing wire 61 is stored inside the wire storage wheel 24020, and a main material of the flexible pushing wire 61 is a Ni—Ti alloy wire with a certain degree of toughness. One end of the connection tube 24016 is connected to the wire driving mechanism 24012, the other end of the connection tube 24016 is connected to a force transducer 24023 on the magazine base 24013, and there is a bending section of the connection tube 24016. A magazine 24014 is fixed into the magazine base 24013. The docking plate 2407 is installed on the docking plate panel 2406. A stylet disposal tube 24017 is fixed on a stylet disposal hole of the docking plate 2407. The door 2402 is closed when the installation is complete.

At the beginning of the surgery, a corresponding count of delivery tubes 24018 are prepared in accordance with the count of puncture needles required for the surgery, and quick connectors 24029 of a plurality of delivery tubes 24018 are sequentially installed into connection holes 24071 on the docking plate 2407, a locking handle 24042 is sequentially rotated to a first gear position, a cam 24034 internally connected with the locking handle 24042 rotates to rotate a connector locking plate 24037, the connector locking plate 24037 is provided with a plurality of convex plates 240371 (connector locking portion) corresponding to the connection holes 24071, and the convex plates 240371 rotate to be embedded in a slot on a side of quick connectors 24029, but not hold against the elastic body 24041, thereby fixing the quick connectors 24029 of the delivery tube 24018 to the docking plate 2407. Then the locking handle 24042 is rotated to a second gear position, the cam 24034 internally connected with the locking handle 24042 rotates to cause the connector locking plate 24037 to continue to rotate, and the convex plate 240371 rotates to press against the elastic body 24041 inside the quick connectors 24429, and the elastic body 24041 simultaneously presses against a stylet inside the delivery tube 24018. Then, the mechanism simultaneously locks all the quick connectors 24429 of the delivery tube 24018 and the stylets inside the delivery tube on the docking plate 2407.

On the other side, an operator holds a puncture needle handle and pierces the puncture needle 18 into a target surgical position of a target object 1002 during the surgery. After the puncture is completed, a needle stylet inside the puncture needle (the needle stylet is very short in length and is used only for puncture) is pulled out, and then the long flexible stylet protruding from the front end of the delivery tube is inserted into the puncture needle to fill the space inside the puncture needle, and then the rear end of the puncture needle is connected to a second quick connector at the front end of the delivery tube. During this process, the long flexible stylet is fixed by the elastic body, so the long flexible stylet would not be withdrawn.

Then, turning the locking handle 24042 along an opposite direction to the first gear position, a spring 24039 connected with the connector locking plate 24037 springs back to cause the convex plate 240371 to loosen the elastic body 24041 and the stylet, but still lock the quick connectors 24029, and the above steps may be repeated until all delivery tubes 24018 are ready for the stylet pulling.

When a user starts the seed implantation system, the machine starts to operate, a motor 24019 controls an internal drum 24010 to rotate, thereby making the stylet pulling mechanism 24011 move to align with a delivery tube 24018. Then the stylet pulling mechanism 24011 starts to work and at the same time the stylet pulling docking mechanism drives the stylet pulling mechanism 24011 to move forward to dock with the tail portion of the stylet, and the stylet pulling mechanism 24011 pull out the stylet inside the delivery tube 24018, and the stylet may be delivered to the stylet storage wheel 24015 (a concave structure) on the rear side of the stylet pulling mechanism 24011, the stylet automatically coil inside the stylet storage wheel 24015 under its own elastic action, and drive the stylet storage wheel 24015 to rotate synchronously. After the stylet is completely pulled out of the delivery tube 24018, the stylet pulling mechanism 24011 returns backwardly. The stylet pulling mechanism 24011 is controlled to move back and forth through a linear movement mechanism, and the linear movement mechanism is one or a combination of a screw nut mechanism, an electric pushing rod, and a rack and pinion mechanism. The motor 24019 controls the drum 24010 to rotate so that the stylet pulling mechanism 24011 is aligned with the recycling tube 24017 (aligned with the recycling hole of the docking plate 2407), and the stylet pulling mechanism 24011 then works to completely spit out the stylet and inserts the stylet into the recycling tube 24017.

Subsequently, the motor 24019 drives the drum 24010 to rotate so that the front end of the wire output channel is aligned to the delivery tube 24018 in which the stylet has just been pulled, and the front end of the wire output channel is docked to the quick connector 24029 of this delivery tube 24018. The front end of the wire output channel is controlled to move back and forth through the implantation docking mechanism, and then the wire driving mechanism 24012 pushes out the flexible pushing wire inside the wire storage wheel 24020, and the flexible pushing wire pushes out a seed inside the magazine 24014 to the docking tube structure 24026 at the front end of the wire output channel, and repeats until the wire output channel is piled up with a specified number of seeds, and then all of the seeds in front of the flexible pushing wire at once are pushed into the delivery tube 24018 until they are delivered into a second puncture needle 101504.

In this scheme, the magazine base 24013 is disposed at the front portion of the wire output channel, a passive measurement wheel is provided inside the magazine base 24013 to press the flexible pushing wire, the friction generated during the movement of the flexible pushing wire drives the passive measurement wheel to rotate, and the rotation angle of the passive measurement wheel is measured by an encoder 24025. A displacement of the flexible pushing wire may be converted by the encoder 24025. Since an encoder module and a measuring wheel are also disposed inside the wire driving mechanism 24012, then the readings of the two encoders may be used to determine whether the passive measurement wheel is slipping, so as to ensure the reliability of a measurement result based on two measurement results, and through reciprocal movements, seeds being pushed reach a specified count. Then the wire driving mechanism 24012 drives the flexible pushing wire to push out the seeds through the delivery tube 24018 to the front end of the puncture needle. In this process, if a resistance subjected by the flexible pushing wire becomes larger during movement, its reaction force may be transmitted to the force sensor 24023 connected with the connection tube 24016 and fed back.

In another embodiment, reflective stripes or color stripes are provided on the flexible pushing wire, and a photoelectric sensor is disposed in the magazine base 24013 or the wire driving mechanism 24012, so that when the relative displacement of the flexible pushing wire and the photoelectric sensor occurs, the reflective stripes or color stripes may synchronously generate a pulse signal, and an actual displacement of the flexible pushing wire may be measured by sampling the pulse signal, and this solution can avoid slippage between the passive measurement wheel and the flexible pushing wire that may affect the measurement accuracy and reliability of the encoder.

Subsequently, a needle pulling rod 24043 is pushed forward until it comes into contact with an outer tube base 101509 of outer tube, at which time a contact signal is sensed by a contact sensor 24027, and the contact sensor 24027 is one or a combination of a force sensor, a mechanical switch, an inductive switch, and a photoelectric switch. Then, as shown in FIG. 24, when a plurality of seeds 101505 reach a position of the second puncture needle 101504 connected with the delivery tube, the wire driving mechanism 24012 may push out the first seed into the human body, and then the needle pulling rod 24043 may continue to push the outer tube base 101509 to move forward, and the other end of the outer tube may be pressed against a surface of the body or a puncture template (which may also be an instrument such as a prostate puncture template, etc.), and when the outer tube base 101509 is pushed forward, an inner tube 101508 moves relative to the outer tube and pulls the second puncture needle 101504 connected at the front end of the inner tube partly out of the human body, and then the wire driving mechanism 24012 may continue to push out a next seed 101505 until the plurality of seeds are implanted respectively through repeated operations.

The wire storage wheel 24020 is a wheel-shape container with a concave inner surface (referring to FIG. 25), the wheel-shape container may rotate freely around an axis thereof, and an edge of the wheel-shape container is a concave structure, a storage region is formed at the concave inner surface of the wheel-shape container. At least one side of the wheel-shape container is provided with an opening, and the flexible pushing wire 61 is guided by a storage guiding tube 24081 to extend into the wheel-shape container through the opening on the side of the wheel-shape container. When the wire driving mechanism 24012 drives the flexible pushing wire to move back and forth, under an elastic action of the flexible pushing wire, the flexible pushing wire is automatically coiled inside a concave inner surface of the wire storage wheel 24020, and the wire storage wheel 24020 rotates along with the back-and-forth movement of the flexible pushing wire, thereby realizing automatic storage.

In other embodiments, a second stopping step 24082 (or spherical protrusion) may be added to the rear end of the flexible pushing wire, and a position of the second stopping step is set to satisfy following principles: when the front end of the flexible pushing wire extends from the delivery tube into the puncture needle and probes out from the front end of the puncture needle by more than a certain length (the length is less than 10 mm), the second stopping step is stuck on the rear step of the storage guiding tube, so as to realize the mechanical position-limiting, which prevents the flexible pushing rod from penetrating further into body tissue, thus ensuring overall safety. Preferably, the second stopping step is an end surface of a stopping tube socketed to the rear end of the flexible pushing wire, or the second stopping step is a welded ball at the rear end of the flexible pushing wire. When the wire driving mechanism 24012 drives the flexible pushing wire to move backward again, the flexible pushing wire is automatically coiled inside the inner concave surface of the wire storage wheel 24020 under the elastic action of the flexible pushing wire, and the wire storage wheel 24020 rotates along with the back and forth movement of the flexible pushing wire, thereby realizing automatic storage.

In other embodiments, a wire connection portion may also be provided on the wire storage wheel 24020, the wire connection portion may be configured to connect and fix the rear end or a middle portion of the flexible pushing wire with the wire storage wheel 24020. A position of the wire connection portion may be set to satisfy following principles: when the front end of the flexible pushing wire extends from the delivery tube into the puncture needle and probes out from the front end of the puncture needle by more than a certain length, the flexible pushing wire is pulled by the wire connection portion so that it is unable to continue to move forward, thereby realizing a mechanical position limiting, which avoids the flexible pushing wire from piercing into the body tissues to cause injuries, and ensuring the overall safety. The wire connection portion adopts a form of threaded connection, bonding, welding, snap connection, and other forms to realize connecting and fixing.

### Embodiment 6

A flexible delivery system for a radioactive source is provided, the parts of this embodiment that have the same structure as those of Examples 1 to 5 are not described in detail, and differences are as follows.

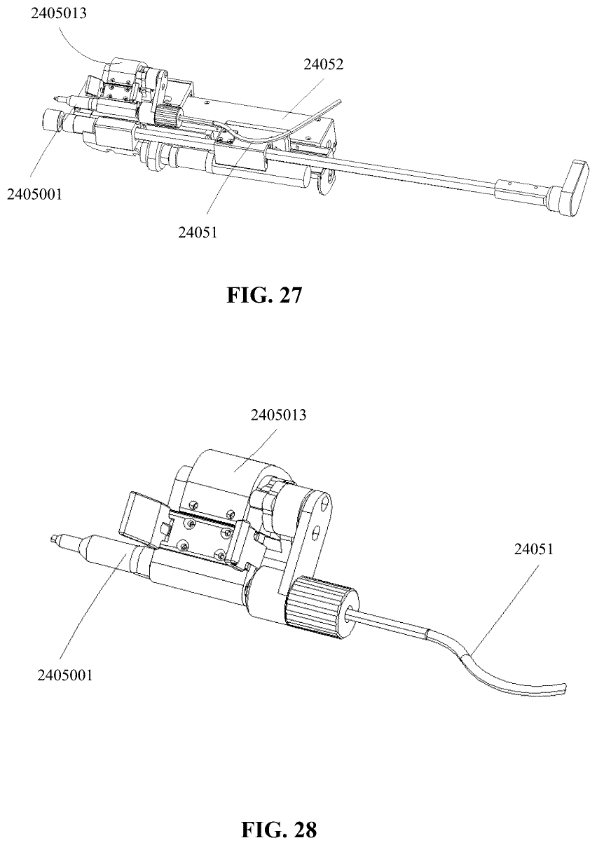

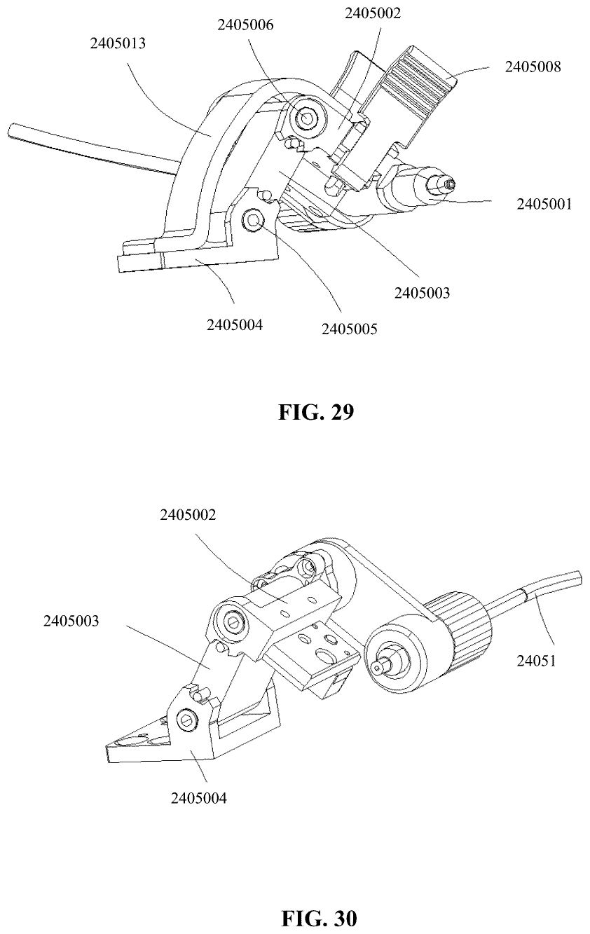

As shown in FIG. 26 to FIG. 32, in this embodiment, the wire driving mechanism 24012, the stylet pulling mechanism 24011, and a docking tube structure 24050 are mounted on the drum 24010.

The magazine 24014 is mounted on the wire driving mechanism 24012, and the wire driving mechanism 24012 is connected to the docking tube structure 24050 through a flexible connection tube 24051, and the docking tube structure 24050 is mounted in the drum 24010 through the implantation docking module 24052 to realize the back-and-forth movement of the docking tube structure 24050 and dock with different flexible delivery tubes on an first connection part.

The docking tube structure 24050 includes a docking head 2405001 and a floating connection mechanism, the docking head 2405001 is mounted on the implantation docking module 24052 by the floating connection mechanism, the floating connection mechanism configured to drive the docking head 2405001 to float in a plane located parallel to the first connection part;

The floating connection mechanism employs two rotary joint modules.

In some embodiments, the floating connection mechanism employs a rotary joint module and a translational module.

In some embodiments, the floating connecting mechanism employs two translational modules, trajectories of the two translation modules are orthogonal to each other or at an angle.

In some embodiments, the floating connection mechanism employs a support seat having a resilient ball hinge, the resilient ball hinge including a third elastic element and a ball hinge, the docking head is mounted at the ball hinge, the ball hinge allows the docking head to be deflected at a certain angle under external force, and the third elastic element is used to realize resetting of the docking head after the external force withdraws.

As shown in FIG. 26 to FIG. 32, when the floating connection mechanism employs two rotary joint modules, the floating connection mechanism includes a first link 2405002, a second link 2405003, and a link seat 2405004, the link seat 2405004 is mounted on the implantation docking module 24052, the second link 2405003 is rotationally set on the link seat 2405004 via a first rotary shaft 2405005, and the rotational resetting of the second link 2405003 is realized via a first elastic element, the first link 2405002 is rotationally set on the second link 2405006 via a second rotary shaft 2405006, and the first link 2405003 is rotationally set on the second link 2405003, and realizes the rotational resetting of the first link 2405002 through the second elastic element, the docking head 2405001 is connected to the first link 2405002 through a quick-lock structure, and the first elastic element and the second elastic element may adopt a torsion spring structure, respectively, and two torsion springs are mounted at the first rotary shaft 2405005 and the second rotary shaft 2405006, respectively. In some embodiments, the first elastic element and the second elastic element are a same elastic plate 2405013, the elastic plate 2405013 having a special shape, one end of the elastic plate 2405013 is fixed to the link seat 2405004 and the other end is fixed to the first link 2405002. When the implantation docking module 24052 drive the docking tube structure to move forward for docking, and the docking head 2405001 is not precisely aligned with the flexible delivery tube on the external connection part, the docking head 2405001 contacts with a conical surface at the connection holes or the rear end of the flexible delivery tube on the external connection part, the first link 2405002 and the second link 2405003 may rotate in a small range so as to realize the adaptive alignment docking. When the implantation docking module 24052 drive the docking tube structure to move backwardly, the first link 2405002 and the second link 2405003 are automatically reset under an action of the first elastic element and the second elastic element so as to be prepared for the next docking.

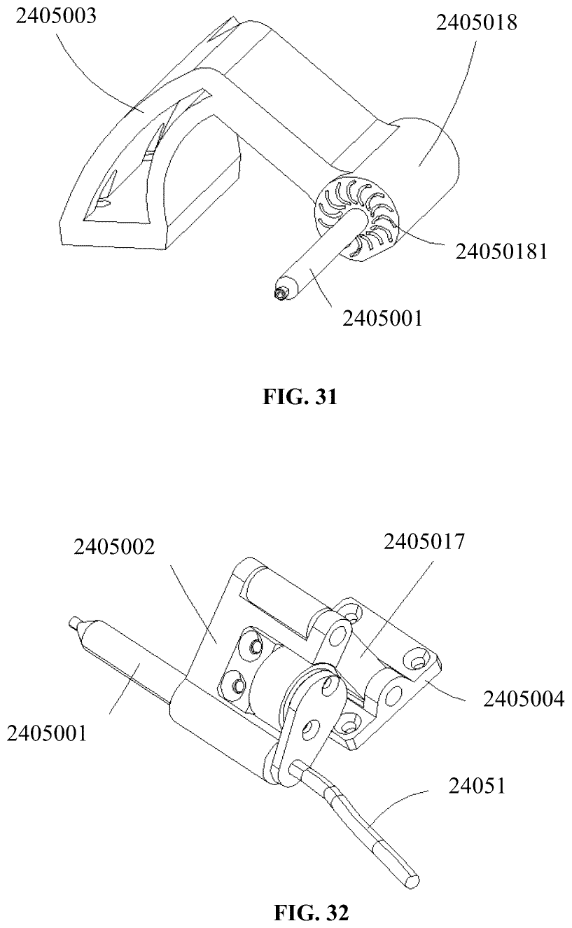

As an alternative, as shown in FIG. 31, when the floating connection mechanism adopts a support seat with an elastic head portion, it includes a support seat 2405017, the support seat 2405017 being mounted on the implantation docking module 24052, the elastic head portion of the support seat 2405017 is an elastic floating ring 2405018 integrally or separately disposed, the docking head 2405001 runs through the elastic floating ring 2405018, the elastic floating ring 2405018 has a deformation slot 24050181 around a periphery of the docking head 2405001, and the deformation slot 24050181 deforms when subjected to a force. By providing the deformation slot 24050181, the docking head 2405001 performs a certain deformation displacement to realize automatic alignment when the implantation docking module 24052 moves forward for docking, which allows a certain alignment error. The deformation slot 24050181 will resume deformation when the implantation docking module 24052 drives the docking tube structure to move backwardly for resetting the docking head 2405001, thereby preparing for a next docking.

As an alternative, as shown in FIG. 32, the docking head 2405001 is fixedly mounted directly to the first link 2405002 without a quick-lock structure.

### Embodiment 7

The present disclosure further proposes a method of using the above-described flexible radioactive source implantation system. The method includes following operations.

Step 1.1: a plurality of flexible delivery tubes are connected with a connection part through a quick connector, respectively, wherein a front end of each flexible delivery tube is connected with a hollow puncture needles inserted into a target body.

Step 1.2: through a planar movement of a first motion platform, a docking tube structure at a front end of a wire output channel is aligned with different quick connectors on the first connection part. And the docking tube structure is docked with different quick connectors through a back-and-forth movement of the first motion platform.

Step 1.3: by a wire driving mechanism, a flexible pushing wire may be driven to push out seeds, or seed strands, or seed strand casings disposed in front of the flexible pushing wire by a radioactive source feeding mechanism to a target position through a flexible delivery tube, and the hollow puncture needles connected in the front end of the flexible delivery tube.

Finally, it should be noted that the above-described embodiments are only specific embodiments of the present invention to illustrate the technical solutions of the present invention, not to limit the technical solutions of the present invention, and the scope of protection of the present invention is not limited thereto, and despite the detailed illustration of the present invention with reference to the aforementioned embodiments. Although the present invention is described in detail with reference to the foregoing embodiments, a person of ordinary skill in the art should understand that any person of skill familiar with the technical field of the present invention can, within the technical scope of the present invention, still make modifications to or easily think of changes in the technical solutions recorded in the foregoing embodiments, or carry out equivalent substitution of some of the technical features therein; and such modifications, changes or substitutions do not make the nature of the corresponding technical solutions depart from the spirit and scope of the technical solutions of the present invention, and the scope of protection of this invention is not limited to this. These modifications, changes or substitutions do not divert the essence of the corresponding technical solutions from the spirit and scope of the embodiments of the present invention, and shall be covered by the scope of protection of the present invention. Therefore, the scope of protection of the present invention shall be stated to be subject to the scope of protection of the claims.

What is claimed is:

1. A flexible delivery system for a radioactive source, comprising:
a main body,
a delivery channel,
a flexible pushing wire, and
a wire driving mechanism, wherein:
the flexible pushing wire is disposed in the delivery channel, the delivery channel being configured to guide the flexible pushing wire to move forward and backward, the wire driving mechanism being configured to drive the flexible pushing wire to move reciprocally along the delivery channel, the flexible pushing wire being configured to push seeds, or seed strands, or seed strand casings to be delivered to a target position through the delivery channel, and
the delivery channel includes a flexible delivery tube and a wire output channel, the flexible delivery tube being connected with the wire output channel, and a length of the flexible pushing wire being greater than 600 mm.

2. The system of claim 1, wherein the flexible pushing wire is a flexible wire with elasticity, the flexible pushing wire is capable of being bent under an external force and being restored to a straight state after the external force is withdrawn, and a material of the flexible pushing wire includes one or a combination of several types, including nickel-titanium alloy, spring steel, and composite materials.

3. The system of claim 1, wherein the wire output channel includes a first flexible section, the first flexible section is a flexible and bendable tube, a length of the first flexible section is greater than 200 mm, and the first flexible section is made of a plastic tube or a medical braided tube.

4. The system of claim 1, wherein one end of the flexible delivery tube is connected with a puncture needle or a quick-connection coupler capable of connecting with the puncture needle, and the quick-connect coupler is connected with the puncture needle through at least one of thread, latch, or adhesive.

5. The system of claim 4, wherein a front end of the puncture needle is provided with a first sharp portion, the puncture needle being made of a bendable and deformable material, an elastic modulus of the bendable and deformable material being less than 50 GPa, and the bendable and deformable material including at least one of nickel-titanium alloy, plastic, or a composite material.

6. The system of claim 1, wherein the flexible delivery tube is provided with an air exhaust structure, the air exhaust structure being configured to equalize air pressures inside and outside of the flexible delivery tube when the flexible pushing wire moves reciprocally along the flexible delivery tube.

7. The system of claim 1, wherein:
the interior of the flexible delivery tube is provided with a flexible stylet,
the flexible stylet is clearance-matched with the flexible delivery tube and is pluggable relative to the flexible delivery tube,
the flexible stylet is a flexible wire with elasticity and capable of being bent under an external force and being restored to a straight state after the external force is withdrawn,
a length of the flexible stylet is greater than 600 mm,
a portion of a rear end of the flexible stylet extends backward from a rear end of the flexible delivery tube, and
a front end of the flexible stylet is located within the interior of the puncture needle connected with a front end of the flexible delivery tube.

8. The system of claim 1, wherein the flexible delivery tube includes a second flexible section, the second flexible section being a bendable and flexible connection tube, a length of the second flexible section being greater than 600 mm, and the second flexible section being made of a plastic tube or a medical braided tube.

9. The system of claim 1, wherein:
the flexible delivery tube includes an inner tube and an outer tube, the outer tube being sleeved outside the inner tube, the inner tube is clearance-matched with the outer tube and is pluggable relative to the outer tube, and
the inner tube is configured to be connected with a puncture needle.

10. The system of claim 9, wherein the inner tube and the outer tube are made of a plastic tube or a medical braided tube.

11. The system of claim 2, wherein a mounting bracket is provided on the wire driving mechanism, the mounting bracket being provided with a wheel-shaped container with a concave inner surface, the flexible pushing wire being coiled on a concave inner side of the wheel-shaped container, and the wheel-shaped container rotates freely on the mounting bracket.

12. The system of claim 1, further comprising:
a radioactive source feeding mechanism, the radioactive source feeding mechanism being disposed on the wire output channel, and the radioactive source feeding mechanism being configured to provide the seeds, or the seed strands, or the seed strand casings through a magazine, wherein:
the seeds, or the seed strands, or the seed strand casings are mounted in a storage slot or a storage hole inside a magazine, and
the seeds, or the seed strands, or the seed strand casings are provided at the front of the flexible pushing wire for feeding through a magazine feeding mechanism mounted on the magazine.

13. The system of claim 12, wherein:
the radioactive source feeding mechanism is a seed magazine or a seed strand magazine, and
the wire driving mechanism includes the seed magazine or the seed strand magazine configured to store radioactive seeds and a seed pushing driving mechanism configured to drive the flexible pushing wire to push the radioactive seeds out of the seed magazine or the seed strand magazine and deliver the radioactive seeds through the delivery channel.

14. The system of claim 1, further comprising:
a first motion platform and a first connection portion, one end of the wire output channel and the first connection portion being mounted on opposite sides of the first motion platform, respectively, wherein:
the first motion platform is configured to realize a relative movement in space between the one end of the wire output channel and the first connection portion;
the first connection portion is connected with a first connection part, the first connection part being provided with a plurality of connection holes, and the first connection portion being at least one of an adhesive connection portion, a welded connection portion, a threaded connection portion, a snap connection portion, or a lock connection portion;
one end of each of the multiple flexible delivery tubes is installed on a different connection hole of the first connection part; and
the wire output channel is configured to deliver the seeds, or the seed strands, or the seed strand casings by docking with one of the plurality of flexible delivery tubes on the first connection part to realize a multi-channel delivery.

15. The system of claim 14, wherein the first motion platform is configured to control a relative movement in space between the one end of the wire output channel and the first connection portion by:
controlling the first connection part to move and the one end of the wire output channel to be stationary,
controlling the first connection part to be stationary and the one end of the wire output channel to move, or
controlling the first connection part to move and the one end of the wire output channel to move.

16. The system of claim 15, wherein:
the first motion platform is configured to realize a relative movement of at least two degrees of freedom between the first connection part and the one end of the wire output channel,
the relative movement includes at least one of:
A. the first connection part is stationary, and the one end of the wire output channel performs a back-and-forth linear movement and a movement within a plane;
B. the first connection part performs a back-and-forth linear movement, and the one end of the wire output channel performs a movement within a plane;
C. the first connection part performs a movement within a plane, and the one end of the wire output channel performs a back-and-forth linear movement; or
D. the first connection part performs a back-and-forth linear movement within a plane, and the one end of the wire output channel is stationary; and
the movement within a plane is one of the following types: a single rotational movement, a single rotational movement combined with a radial linear movement, a double-joint rotational movement, or a double linear movement along X and Y axes.

17. The system of claim 16, wherein:
the first motion platform includes a back-and-forth movement module, a rotational movement module, and a radial movement module, and the first motion platform realizes a movement of three degrees of freedom of the one end of the wire output channel in space through a one-direction rotational movement and a two-direction linear movement; or
the first motion platform includes the back-and-forth movement module and the rotational movement module, and the first motion platform realizes a movement of the one end of the wire output channel in space through a one-direction rotational movement and a one-direction linear movement; or
the first motion platform includes the back-and-forth movement module, a left-and-right movement module, and an up-and-down movement module, and the first motion platform realizes the movement of three degrees of freedom of the one end of the wire output channel in space through a three-direction linear movement; or
the first motion platform is a multi-jointed robotic arm driving the one end of the wire output channel to freely move and to be positioned in a three-dimensional space.

18. The system of claim 17, wherein when the first motion platform includes the back-and-forth movement module and the rotational movement module, the first motion platform is in a shape of a drum, the wire driving mechanism is disposed inside the drum, the rotational movement module drives the drum to rotate around an axis of the drum, and the back-and-forth movement module is disposed inside the drum for driving the one end of the wire output channel to move forward and backward.

19. The system of claim 18, wherein:
the plurality of flexible delivery tubes are secured to the first connection part through a quick connector,
the wire driving mechanism and a docking tube structure are mounted on the first motion platform,
when the wire driving mechanism and the docking tube structure are connected through a flexible connection tube, the flexible connection tube is a portion of the wire output channel, and an outlet of the docking tube structure is a terminal end of the wire output channel,
the docking tube structure is inserted into a tapered hole on the quick connector to realize docking, and
the docking tube structure adopt a floating docking tube structure for floating within a range to realize adaptive alignment docking.

20. A method for using a flexible delivery system for a radioactive source, comprising:
connecting a plurality of flexible delivery tubes with a connection part through a quick connector, respectively, wherein a front end of each of the plurality of flexible delivery tubes is connected with one of a plurality of hollow puncture needles inserted into a target body;
positioning, through a movement of a first motion platform, a docking tube structure at a terminal end of a wire output channel and different quick connectors on the first connection part, and docking, through a back-and-forth movement of the first motion platform, the docking tube structure at the terminal end of the wire output channel with the different quick connectors on the first connection part; and
driving, by a wire driving mechanism, a flexible pushing wire to push out seeds, or seed strands, or seed strand casings disposed in front of the flexible pushing wire by a radioactive source feeding mechanism to a target position through a flexible delivery tube and implanting the seeds, or the seed strands, or the seed strand casings into the target body through the plurality of hollow puncture needles.
